(19) United States
(12) Patent Application Publication
(10) Pub. No.: US 2025/0143896 A1
(43) Pub. Date: May 8, 2025

(54) SPLANCHNIC FLOW RESTRICTOR VALVES

(71) Applicant: EDWARDS LIFESCIENCES CORPORATION, Irvine, CA (US)

(72) Inventors: Michael G. Valdez, Riverside, CA (US); Arnold Cruz Tuason, Claremont, CA (US); Tiana (formerly Thu Thi Anh) Tran, Westminster, CA (US); Emil Karapetian, Huntington Beach, CA (US); Roy Shitrit, Zichron Yaakov (IL)

(21) Appl. No.: 19/015,418

(22) Filed: Jan. 9, 2025

Related U.S. Application Data
(63) Continuation of application No. PCT/US2023/071013, filed on Jul. 26, 2023.
(60) Provisional application No. 63/482,275, filed on Jan. 30, 2023.
(60) Provisional application No. 63/370,165, filed on Aug. 2, 2022.

(51) Int. Cl. A61F 2/82

(52) U.S. Cl. CPC A61F 2/82; A61F 2220/0025; A61F 2230/0067; A61F 2230/0071; A61F 2230/0091

(57) ABSTRACT

A medical implant for managing blood flow through a blood vessel comprises a stent body forming a proximal portion and a tapered distal end, the tapered distal end being angled relative to the proximal portion to face a direction of blood flow and limit blood flow into an inner lumen of the stent body, and the tapered distal end having a flexible structure to allow the tapered distal end to bend and straighten in response to blood flow, and a covering extending along the stent body.

### BACKGROUND

This application is a continuation of International Patent Application No. PCT/US2023/071013, filed Jul. 26, 2023, which claims the benefit of U.S. Provisional Application No. 63/370,165, filed Aug. 2, 2022, and of U.S. Provisional Application No. 63/482,275, filed Jan. 30, 2023, the disclosures of which are hereby incorporated by reference in its entirety.

### BACKGROUND

The present invention relates generally to the field of medical devices and procedures.

Redistribution of blood from the splanchnic venous circulation to the inferior vena cava (IVC) can contribute to increases in central venous pressure (CVP), pulmonary artery pressure, and/or pulmonary capillary wedge pressure (PCWP), particularly during periods of elevated sympathetic tone (e.g., exercise) in heart failure patients.

### SUMMARY

Some implementations of the present disclosure relate to a medical implant for managing blood flow through a blood vessel, the medical implant including: a plug; a mounting ring; and one or more arms interconnecting the plug and the mounting ring, wherein the one or more arms are configured to allow movement of the plug relative to the mounting ring.

In some aspects, the techniques described herein relate to a medical implant, wherein the blood vessel is an inferior vena cava.

In some aspects, the techniques described herein relate to a medical implant, wherein the plug includes a conical proximal end.

In some aspects, the techniques described herein relate to a medical implant, wherein the plug includes a rounded distal end.

In some aspects, the techniques described herein relate to a medical implant, wherein the plug includes a midsection between the proximal end and the distal end, and wherein the midsection has a greater width than the proximal end and the distal end.

In some aspects, the techniques described herein relate to a medical implant, wherein the proximal end is disposed proximally to the mounting ring in a default state of the one or more arms.

In some aspects, the techniques described herein relate to a medical implant, wherein the one or more arms form bends.

In some aspects, the techniques described herein relate to a medical implant, wherein the one or more arms are shape-set to a default state.

In some aspects, the techniques described herein relate to a medical implant, wherein the one or more arms are configured to elastically deform in response to blood pressure against a distal end of the plug.

In some aspects, the techniques described herein relate to a medical implant, wherein the one or more arms are configured to allow the plug to at least partially enter a lumen of the mounting ring.

In some aspects, the techniques described herein relate to a medical implant, wherein the one or more arms are configured to allow the plug to pass fully through the lumen of the mounting ring.

In some aspects, the techniques described herein relate to a medical implant, wherein at least one of the one or more arms has a constant width.

In some aspects, the techniques described herein relate to a medical implant, wherein at least one of the one or more arms includes a proximal end and a distal end, and wherein the proximal end is wider than the distal end.

In some aspects, the techniques described herein relate to a medical implant, wherein the at least one of the one or more arms has a tapered width.

In some aspects, the techniques described herein relate to a medical implant, wherein the at least one of the one or more arms has a step-like width.

In some aspects, the techniques described herein relate to a medical implant for managing blood flow through a blood vessel, the medical implant including: a stent body having an inner lumen; and a restrictor valve having a tapered distal end extending at least partially over the inner lumen of the stent body.

In some aspects, the techniques described herein relate to a medical implant, wherein the tapered distal end of the restrictor valve is configured to face a direction of blood flow through the blood vessel.

In some aspects, the techniques described herein relate to a medical implant, wherein the tapered distal end of the restrictor valve is configured to at least partially flatten in response to increase blood pressure through the blood vessel.

In some aspects, the techniques described herein relate to a medical implant, wherein the blood vessel is an inferior vena cava.

In some aspects, the techniques described herein relate to a medical implant, wherein the tapered distal end of the restrictor valve forms an orifice into the inner lumen of the stent body.

In some aspects, the techniques described herein relate to a medical implant, wherein the orifice is at a central position of the restrictor valve.

In some aspects, the techniques described herein relate to a medical implant, wherein the tapered distal end of the restrictor valve is configured to reduce a size of the orifice in response to increased blood pressure through the blood vessel.

In some aspects, the techniques described herein relate to a medical implant, wherein the tapered distal end of the restrictor valve is configured to fully close the orifice in response to increased blood pressure through the blood vessel.

In some aspects, the techniques described herein relate to a medical implant, wherein the restrictor valve includes one or more bypass apertures to allow blood flow through the restrictor valve.

In some aspects, the techniques described herein relate to a medical implant, wherein the one or more bypass apertures are disposed at the tapered distal end of the restrictor valve.

In some aspects, the techniques described herein relate to a medical implant, wherein the one or more bypass apertures are disposed at a proximal portion of the restrictor valve.

In some aspects, the techniques described herein relate to a medical implant, wherein the one or more bypass apertures are disposed at a transition between a proximal portion of the restrictor valve and the tapered distal end of the restrictor valve.

In some aspects, the techniques described herein relate to a medical implant, wherein the one or more bypass apertures are configured to increase in size in response to flattening of the tapered distal end of the restrictor valve.

In some aspects, the techniques described herein relate to a medical implant, wherein the tapered distal end of the restrictor valve includes two or more leaflets.

In some aspects, the techniques described herein relate to a medical implant, wherein the two or more leaflets at least partially overlap.

In some aspects, the techniques described herein relate to a medical implant, wherein the restrictor valve includes a covering extending at least partially over an outer surface of the stent body.

In some aspects, the techniques described herein relate to a medical implant, wherein the restrictor valve includes a covering extending at least partially along an inner surface of the stent body.

In some aspects, the techniques described herein relate to a medical implant, wherein the stent body includes one or more curved arms configured to support the tapered distal end of the restrictor valve.

In some aspects, the techniques described herein relate to a medical implant, further including a plug coupled to the stent body via a tether.

In some aspects, the techniques described herein relate to a medical implant, wherein the tether includes a coiled wire.

In some aspects, the techniques described herein relate to a medical implant, wherein the tether is configured to hold the plug distally from the stent body.

In some aspects, the techniques described herein relate to a medical implant, wherein the tether is configured to allow the plug to move towards the stent body in response to increase blood pressure through the blood vessel.

In some aspects, the techniques described herein relate to a medical implant, wherein the plug is configured to fit into an orifice at the tapered distal end of the restrictor valve.

In some aspects, the techniques described herein relate to a medical implant, wherein the plug includes a conical proximal end.

In some aspects, the techniques described herein relate to a medical implant, wherein the plug includes a rounded distal end.

In some aspects, the techniques described herein relate to a medical implant, wherein the restrictor valve is coupled to the stent body via a tether.

In some aspects, the techniques described herein relate to a medical implant, wherein the restrictor valve includes a bowl forming a spherical cap extending towards the inner lumen of the stent body and a concave interior facing a direction of blood flow through the blood vessel.

In some aspects, the techniques described herein relate to a medical implant, wherein the restrictor valve includes crossing arms supporting the bowl.

In some aspects, the techniques described herein relate to a medical implant, wherein the stent body includes a stopper configured to prevent at least a portion of the crossing arms from entering the inner lumen of the stent body.

In some aspects, the techniques described herein relate to a method including percutaneously delivering, via a catheter, a medical implant for managing blood flow through a blood vessel, the medical implant including: a stent body having an inner lumen; and a restrictor valve having a tapered distal end extending at least partially over the inner lumen of the stent body.

For purposes of summarizing the disclosure, certain aspects, advantages, and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loudspeakers, headphones, pressure transducers, temperature transducers, or using any combination of suitable technologies.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 provides a schematic representation of portions of the splanchnic circulation.

FIG. 2 provides another schematic representation of the splanchnic circulation, illustrating blood flow from the aorta to the inferior vena cava (IVC).

FIG. 3 illustrates portions of the splanchnic venous circulation acting as a blood reservoir between the aorta and the IVC.

FIG. 4 illustrates an example medical implant in a default form and disposed within a blood vessel (e.g., a hepatic vein) of a heart, in accordance with one or more examples.

FIG. 5 illustrates an example medical implant in a compressed form and disposed within a blood vessel of a heart, in accordance with one or more examples.

FIGS. 6A-6D illustrate an example occlusion device disposed within a blood vessel (e.g., a hepatic vein) in accordance with one or more examples.

FIGS. 7A and 7B illustrate an example occlusion device comprising one or more leaflets and disposed within a blood vessel (e.g., a hepatic vein) in accordance with one or more examples.

FIGS. 8A-8C illustrate another example occlusion device comprising one or more leaflets and disposed within a blood vessel (e.g., a hepatic vein) in accordance with one or more examples

FIG. 9 illustrates an example plug configured or plugging and/or closing an opening of an occlusion device in accordance with one or more examples.

FIG. 10A illustrates an example occlusion device in a blood vessel and comprising a plug in a default and/or expanded form in accordance with one or more examples.

FIG. 10B illustrates the example occlusion device in a compressed form in accordance with one or more examples.

FIGS. 11A-11C illustrate another example occlusion device configured to selectively occlude a blood vessel in accordance with one or more examples.

FIGS. 12A-12C illustrates an example occlusion device in a blood vessel and comprising a plug in a default and/or expanded form in accordance with one or more examples.

FIGS. 13A-13C illustrates an example occlusion device in a blood vessel and comprising a plug in accordance with one or more examples.

FIG. 14 illustrates an arm having a generally constant width.

FIG. 15 illustrates an arm having a single-tiered variable width.

FIG. 16 illustrates an arm having a gradual and/or variable width.

FIG. 17 illustrates an arm having a multi-tiered variable width.

FIG. 18 provides a flowchart illustrating an example process of delivering and/or anchoring the various occlusion devices described herein.

### DETAILED DESCRIPTION

The headings provided herein are for convenience only and do not necessarily affect the scope or meaning of the claimed invention.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

### Overview

The following includes a general description of human cardiac anatomy that is relevant to certain inventive features and examples disclosed herein and is included to provide context for certain aspects of the present disclosure.

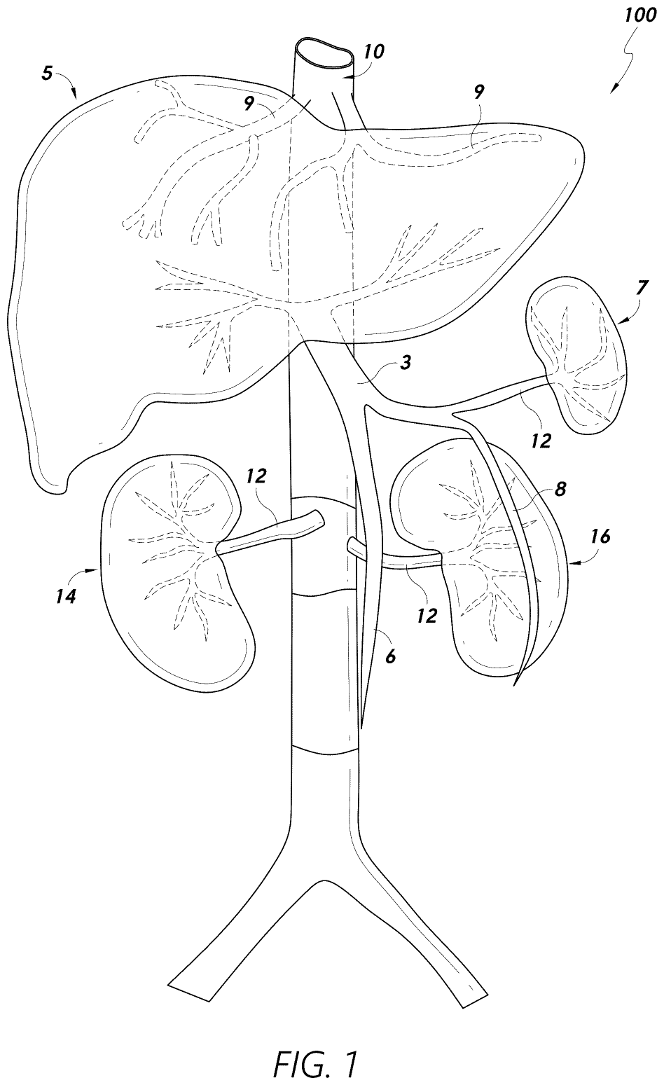

FIG. 1 provides a schematic representation of portions of the splanchnic circulation 100. The term “splanchnic circulation” refers to blood flow originating from the celiac, superior mesenteric, and inferior mesenteric arteries to the abdominal gastrointestinal organs. The splanchnic circulation 100 receives approximately 25% of the cardiac output and holds a similar percentage of the total blood volume under normal conditions. The splanchnic circulation 100 can act as a site of cardiac output regulation and/or as a blood reservoir. Multiple regulatory pathways are involved in the distribution of the splanchnic circulation.

Total flow to the splanchnic viscera is controlled by resistance vessels in the mesenteric and hepatic arterial systems. The venous effluents from the splanchnic viscera converge to form the portal vein 3, which supplies approximately 75% of the total blood supply to the liver 5. The portal blood not only is high in substrate concentrations resulting from intestinal absorption but also tends to contain bacteria and endotoxin.

Renal veins 12 drain blood from the right kidney 14 and left kidney 16 and connect to the inferior vena cava 10 (IVC). The superior mesenteric vein 6 is a major venous tributary of the abdominal cavity that lies laterally to the superior mesenteric artery and serves to drain the vast majority of the organs of the abdominal cavity. The inferior mesenteric vein 8 drains blood from the large intestine. The splenic vein 12 is a blood vessel that drains blood from the spleen, the stomach fundus, and part of the pancreas.

The portal vein 3 receives blood from the stomach, intestines, pancreas, and spleen 7 and carries it into the liver 5 through the porta hepatis. The porta hepatis serves as the point of entry for the portal vein 3 and the proper hepatic artery and is the point of exit for the bile passages.

Following processing of the blood by the liver 5, the blood collects in the central vein at the core of the lobule. Blood from these central veins ultimately converges in the right and left hepatic veins 9, which exit the superior surface of the liver 5 and empty into the IVC 10 to be distributed to the rest of the body.

The splanchnic venous circulation 100 is highly compliant and can act as a blood reservoir that can be recruited in order to support the need for increased stressed blood volume during periods of elevated sympathetic tone, such as exertion, in order to support increased cardiac output and vasodilation of peripheral vessels supporting active muscles. However, heart failure patients can have multiple comorbidities that prevent them from using that additional blood volume. Such comorbidities can include chronotropic incompetence, inability to increase stroke volume, and/or peripheral microvascular dysfunction. This can lead to venous congestion and/or abrupt rises in pulmonary capillary wedge pressure (PCWP).

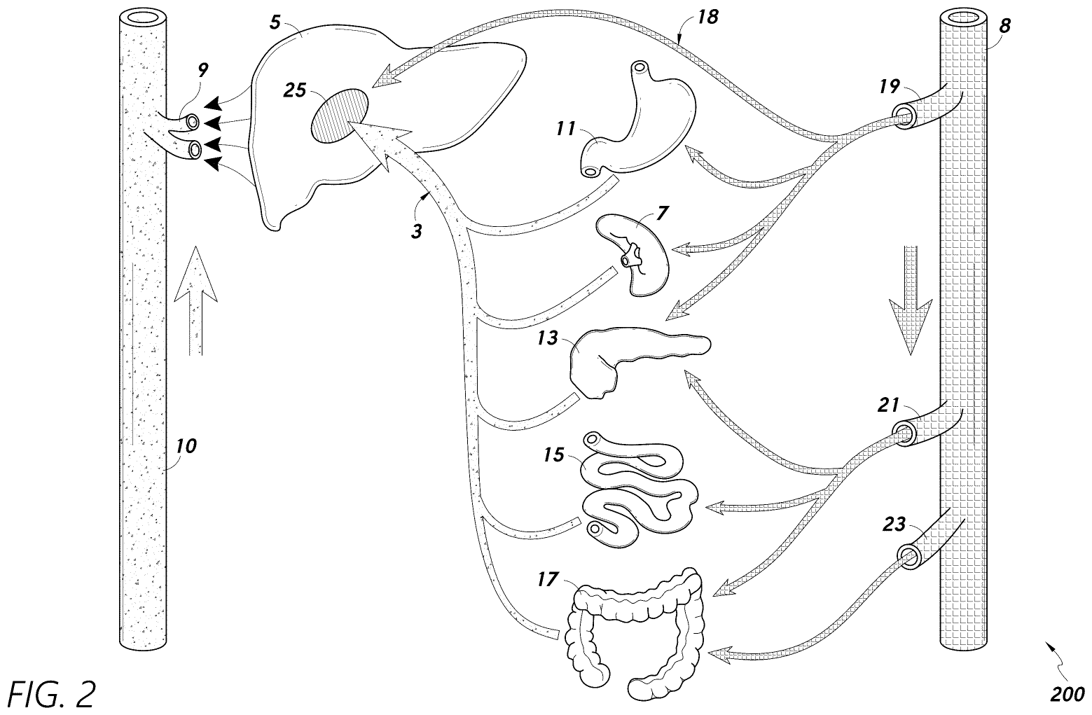

FIG. 2 provides another schematic representation of the splanchnic circulation 200, illustrating blood flow from the aorta 8 to the IVC 10. Blood travels from the aorta 8 to the abdominal gastrointestinal organs including the stomach 11, liver 5, spleen, 7, pancreas 13, small intestine 15, and large intestine 17. The splanchnic circulation 200 comprises three major branches of the abdominal aorta 9, including the coeliac artery 19, the superior mesenteric artery 21 (SMA), and the inferior mesenteric artery 23 (IMA). The hepatic portal circulation (e.g., the hepatic artery 18 and/or portal vein 3) delivers the majority of blood flow to the liver 5.

The coeliac artery 19 is the first major division of the abdominal aorta 8, branching at T12 in a horizontal direction ˜1.25 cm in length. It shows three main divisions such as the left gastric artery, common hepatic artery 18, and splenic artery and is the primary blood supply to the stomach 11, upper duodenum, spleen 7, and pancreas 13.

The SMA 21 arises from the abdominal aorta 8 anteriorly at L1, usually 1 cm inferior to the coeliac artery 19. The five major divisions of the SMA 21 are the inferior pancreaticoduodenal artery, intestinal arteries, ileocolic, right colic, and middle colic arteries. The SMA 21 supplies the lower part of the duodenum, jejunum, ileum, caecum, appendix, ascending colon, and two-thirds of the transverse colon. It is the largest of the splanchnic arterial vessels delivering >10% of the cardiac output and therefore has significant implications for embolic mesenteric ischaemia.

The IMA 23 branches anteriorly from the abdominal aorta 8 at L3, midway between the renal arteries and the iliac bifurcation. The main branches of the IMA 23 are the left colic artery, the sigmoid branches, and the superior rectal artery. It forms a watershed with the middle colic artery and supplies blood to the final third of the transverse colon, descending colon, and upper rectum.

Blood flow is conveyed into the liver 5 via the portal vein 3 into sinusoids 25 of the liver 5. The hepatic veins 9 convey the blood from the liver 5 to the IVC 10.

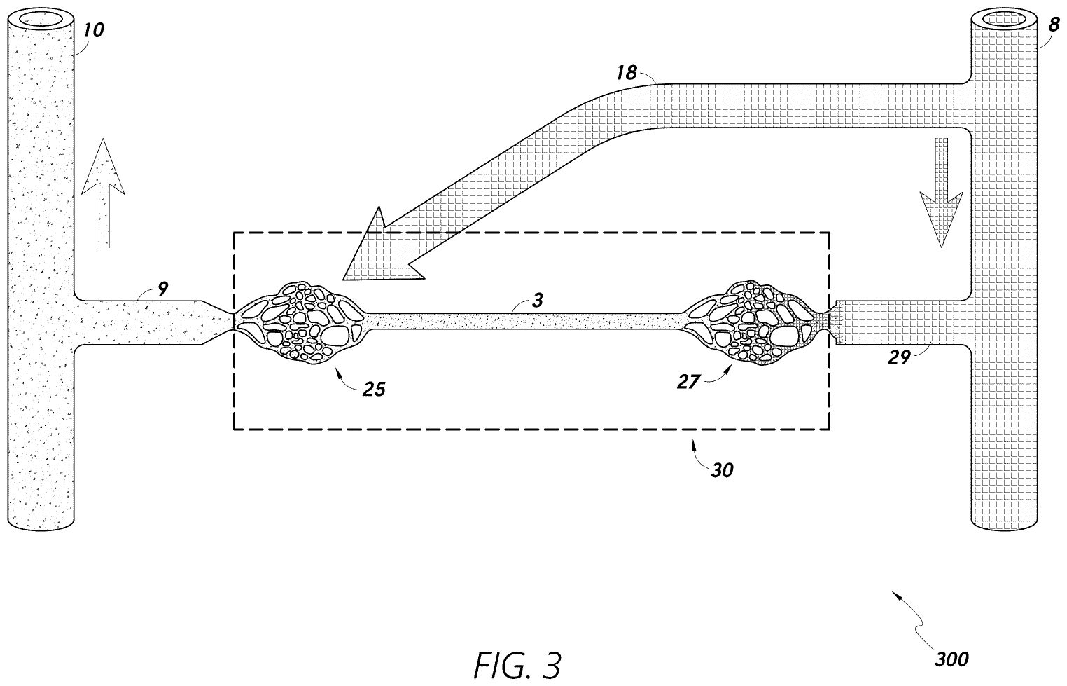

FIG. 3 illustrates portions of the splanchnic venous circulation 300 acting as a blood reservoir 30 between the aorta 8 and the IVC 10. The portal vein 30 conveys blood between the splanchnic organs 27 (e.g., the stomach, spleen, etc.) and the liver sinusoids 25. The liver sinusoids 25 also receive blood from the hepatic artery 18. The splanchnic organs 27 receive blood from the aorta 8 via various splanchnic arteries 29 (e.g., the SMA, IMA, etc.). The amount of blood contained in the portal vein 3 at any given time can be variable.

For some patients (especially patients experiencing heart failure) fluid redistribution from the splanchnic venous reservoir 30 to the IVC 10 and/or stressed blood volume can contribute to increases in central venous pressure (CVP), pulmonary artery pressure, and/or PCWP. This can be especially problematic during periods of elevated sympathetic tone, such as exertion, and/or can lead to pulmonary congestion that can impact a patient's quality of life and/or can lead to acute decompensation.

The splanchnic venous circulation 300, and particularly the portal vein 3, can advantageously provide a blood reserve to support the need for increased stressed blood volume during periods of elevated sympathetic tone. Because blood flow from the splanchnic venous circulation 300 is directed through the hepatic veins 9 and into the IVC 10, devices placed into the hepatic veins 9 and/or IVC 10 to limit blood flow can allow the reservoir 30 to expand with increased blood volume.

Examples described herein can relate to devices and/or methods that can advantageously limit, stagnate, and/or impede blood flow into the IVC 10 from the hepatic veins 9 to increase the pressure gradient between the IVC 10 and the liver and/or splanchnic venous circulation 300. In some examples, one or more flow-regulating implants may be configured for placement at least partially within the hepatic veins 9 and/or IVC 10 and/or at one or more junctions between the hepatic veins 9 and the IVC 10. As a result, blood flowing from the splanchnic venous reservoir 30 into the hepatic veins 9 can be slowed to increase blood volume in the splanchnic venous reservoir 30.

Patients with Heart Failure with Preserved Ejection Fraction (HFpEF) can be hospitalized due to increased pressures in the left atrium. Increased left atrial pressures can be transmitted into the pulmonary circulation and ultimately can lead to lung congestion and dyspnea. Volume redistribution from the splanchnic vascular network 300 into circulation can contribute to patient decompensation.

The splanchnic network 300 is the body's largest blood reservoir and can hold up to about 20% of the body's total blood volume. During exertion or increased sympathetic autonomic nervous system activity, a significant portion of this blood is redistributed into circulation, causing an acute increase in cardiac preload. In healthy physiology, this elevated preload causes the ventricle to fill more and follows the Frank Starling curve, enabling the ventricle to increase its stroke volume.

However, in patients suffering from HFpEF, the elevated preload cannot increase ventricular filling due to diastolic dysfunction (ventricular stiffness). As a result, elevated preload backs up into the left atrium and the lungs, leading to pulmonary congestion, shortness of breath, and/or hospitalization.

The splanchnic circulation 300 redistributes blood volume into circulation during elevation of sympathetic tone (stress response, exertion, etc.). This extra volume increases preload leading to higher contractility (Frank-Starling Law) and aids in supplying the elevated demand for cardiac output. For patients with HFpEF, this extra blood volume in circulation cannot be accommodated by the failing ventricle with diastolic dysfunction. This causes a backup of pressure into the left atrium leading to pulmonary congestion.

Splanchnic blood redistribution occurs through the hepatic veins. Limiting redistribution of blood into circulation can help prevent elevated preload, pulmonary congestion, shortness of breath and heart failure hospitalizations.

Some approaches to reducing volume redistribution can involve placing fixed orifice flow restrictors. Restricting the flow from the hepatic veins can be beneficial in preventing volume redistribution. However, too much restriction can cause hepatic congestion. It may be advantageous to modulate the response and increase restriction only during volume redistribution.

Some examples presented herein relate to methods and devices for increasing the restriction of blood flow coming from the hepatic veins and IVC into the right atrium as blood pressure increases. In some instances, a device comprises a stent and/or similar device placed in the IVC. The device can have multiple leaflets and/or a tapered end portion, similar to a prosthetic heart valve. The leaflets and/or end portion may be configured to at least partially close in response to increased blood pressure and/or open in response to decreased blood pressure.

Some devices described herein can be disposed in the IVC such that leaflets and/or end portions of the devices can be disposed at the bottom of the device and/or facing the inflow of blood as it travels upward (e.g., into the right atrium).

Some devices described herein can be implanted using a transcatheter and/or transvenous approach. Devices can be biased towards an open position and/or can be configured to modify/adjust to a partially and/or fully closed state in response to changes in blood pressure. The devices can thus be configured to return to an open state as blood pressure lowers.

Some examples described herein related to devices and/or processes for limiting redistribution of blood (e.g., from the IVC). Example devices can include a stent body forming and/or comprising a distal end that faces a direction of blood flow within a blood vessel (e.g., the IVC). For example, blood flowing through the blood vessel may first contact the distal end of the device before contacting other parts of the device. The stent body and/or distal end of the stent body can form an orifice to allow blood flow through and/or into a lumen of the stent body. The distal end of the stent body may be configured to default to an open state in which the orifice through the distal end may be open and/or relatively large. The distal end may be configured to naturally move/adjust in response to increases in blood pressure to a closed state in which the orifice through the distal end is blocked and/or relatively small.

In some examples, the distal end of the stent body can comprise two or more leaflets extending from the stent body and/or configured to extend around and/or over the orifice situated between and/or in the middle of the leaflets. The distal end can comprise a tapered and/or conical sheet of material. In some examples, the distal end comprises one or more bypass openings to allow some blood flow through the leaflets and/or sheet. The one or more bypass openings may be situated at intersection points between the leaflets/sheet and the stent body and increase in size as the leaflets/sheet close.

In some example devices described herein, the distal of the stent body can comprise a plug configured to be held distally from the stent body in the open state by a coil extending from the stent body. The plug and/or coil may be configured to be moved towards the stent body in response to increase blood pressure against the plug and/or coil.

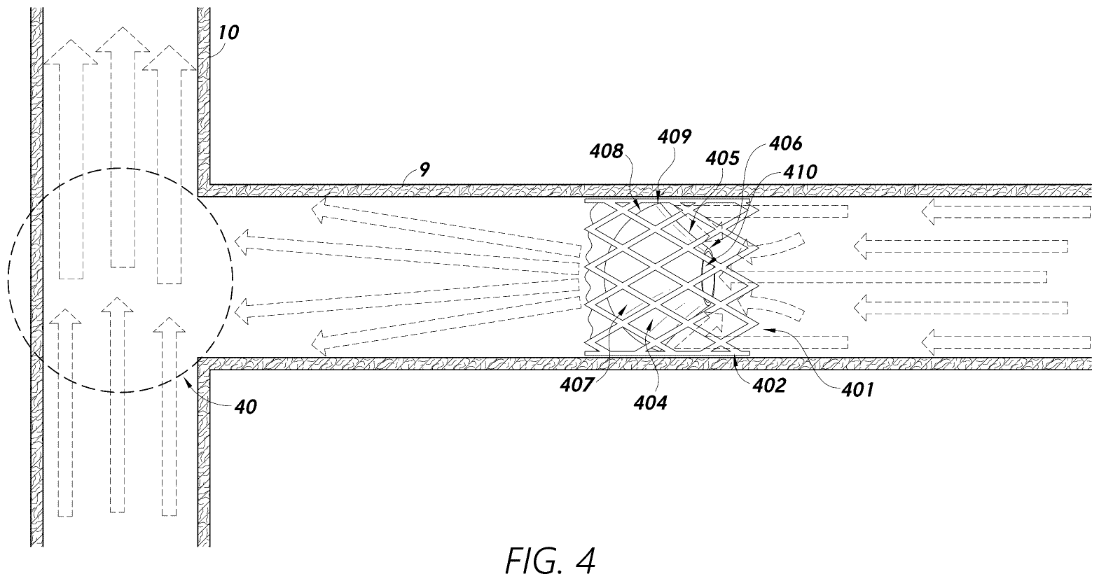

FIG. 4 illustrates an example medical implant 401 in a default (e.g., expanded and/or resting) form and disposed within a blood vessel 9 (e.g., a hepatic vein) of a heart, in accordance with one or more examples. The blood vessel 9 may provide a conduit for blood flow from the splanchnic reservoir into the IVC 10 and/or other blood vessel. As indicated by arrows in FIG. 4, blood may flow from the right side of the page to the left and/or towards the IVC 10. While the implant 401 is shown implanted within a hepatic vein 9, example implants 401 described herein may be suitable for use in other blood vessels and/or chambers of a heart.

The medical implant 401 may comprise a stent body 402 and/or a valve 404. The term “valve” is used herein in accordance with its plain and ordinary meaning and may refer to any device configured to passively, selectively, and/or otherwise manage, restrict, occlude, and/or obstruct blood flow through a blood vessel and/or chamber of a heart. A valve may include any means for occluding, occlusion element, means for obstructing, obstruction element, means for restricting, and/or restriction element. While the term “valve” is used in FIG. 4 to refer to a restriction element situated within a stent body, in some examples herein the term “valve” may refer to a device comprising both a stent body and a restriction element.

In some examples, the valve 404 may be similar to a prosthetic heart valve. For example, the valve 404 may comprise one or more leaflets and/or may selectively form an opening 410 for blood flow through the valve 404 and/or stent body 402 and/or may close the opening 410 during periods of increased blood pressure. The opening 410 of the valve 404 may face a direction of blood flow. For example, the opening 410 may be formed at a distal end 408 of the valve 404 that is distal from a proximal end 406 of the valve 404. In some examples, the proximal end 406 may form a larger opening and/or orifice than the opening 410 formed at the distal end 408 based at least in part on a conical shape of the valve 404.

In some examples, the implant 401 may be biased and/or defaulted in an at least partially open form, as shown in FIG. 4. For example, the implant 401 may be implanted in a form in which the opening 410 provides at least partial and/or relatively high blood flow into and/or through the valve 404. In response to blood flow and/or pressure changes in the vessel 9, the implant 401 may be configured to adjust and/or deform such that the opening 410 provides relatively low blood flow into and/or through the valve 404.

The valve 404 may comprise a distal portion 405 configured to adjust and/or deform to regulate blood flow into and/or through the valve 404. In some examples, the distal portion 405 may have a conical and/or tapered form with a decreasing diameter relative to a diameter of a proximal portion 407 of the valve 404 and/or to a diameter of the stent body 402.

In some examples, the distal portion 405 may comprise a generally conical sheet of material with an opening 410 and/or orifice formed through a center portion of the distal portion 405. In other examples, the distal portion 405 may comprise two or more overlapping and/or adjacent leaflets extending around the opening 410. The distal portion 405 may comprise a generally flexible material configured to fold, bend, and/or otherwise deform in response to increased blood pressure. In some examples, the distal portion 405 may comprise multiple segments and/or leaflets configured to interconnect and/or at least partially overlap. In response to increased pressure, segments of the distal portion 405 may adjust to a greater amount of overlap and/or the distal portion 405 may relax and/or at least partially stretch and/or flatten.

Blood flow through the vessel 9 may be configured to press against an outer surface of the distal portion 405 and/or of the valve 404. For example, the distal portion 405 may be angled such that at least part of the distal portion 405 faces the direction of flow through the vessel 9. In some examples, the distal portion 405 may be configured to be responsive to blood flow above a threshold pressure level. High blood pressure through the vessel 9 may be configured to press against the outer surface of the distal portion 405 and/or to compress, stretch, flatten, and/or otherwise move the distal portion 405 such that the opening 410 decreases in size.

The stent body 402 may have a cylindrical and/or tubular form and/or may form an inner lumen extending through the stent body 402. The inner lumen may be generally cylindrical in shape and/or may be configured to be disposed coaxially with the vessel 9. In some examples, the tapered distal portion 405 of the valve 404 may be disposed at least partially within and/or beyond the inner lumen of stent body 402. The opening 410 and/or orifice formed by the distal portion 405 may be at a central portion of the valve 404 and/or may be generally coaxial with the inner lumen of the stent body 402. In some examples, the tapered distal portion 405 of the valve 404 may be configured to extend at least partially over the inner lumen of the stent body 402. The distal portion 405 of the valve 404 may be configured to reduce the size of the opening 410 in response to increased blood pressure through the blood vessel 9. In some examples, the distal portion 405 may be configured to fully close the opening 410 in response to increased blood pressure through the blood vessel 9.

In some examples, the valve 404 may comprise sides 409 configured to contact and/or form a seal against the vessel 9. For example, the valve 404 may form a generally circular cross-section and/or may form a generally circular area of contact with the vessel 9.

FIG. 4 illustrates blood flow into the blood vessel 9 from one or more splanchnic arteries through the splanchnic reservoir and finally into the IVC 10. While only a single blood vessel 9 is shown in FIG. 4 for illustrative purposes, multiple blood vessels (e.g., hepatic veins) may convey blood from the reservoir into the IVC 10. The blood vessel 9 may feed into a junction portion 40 of the IVC 10. Accordingly, to limit blood flow into the IVC 10, one or more flow-limiting implants 401 may be configured for placement within the blood vessel 9 and/or at least partially within the junction portion 40 of the IVC 10.

The present disclosure provides methods and devices (including various medical implants) for managing blood flow within a human body. The term “implant” is used herein according to its plain and/ordinary meaning and may refer to any medical implant, frame, valve, shunt, stent, anchor, and/or similar devices for use in treating various conditions in a human body. Implants may be delivered percutaneously and/or via catheter (i.e., transcatheter) for various medical procedures and may have a generally sturdy and/or flexible structure. The term “catheter” is used herein according to its broad and/ordinary meaning and may include any tube, sheath, steerable sheath, steerable catheters, and/or any other type of elongate tubular delivery device comprising an inner lumen configured to slidably receive instrumentation, such as for positioning within an IVC and/or hepatic vein, including for example delivery catheters and/or cannulas.

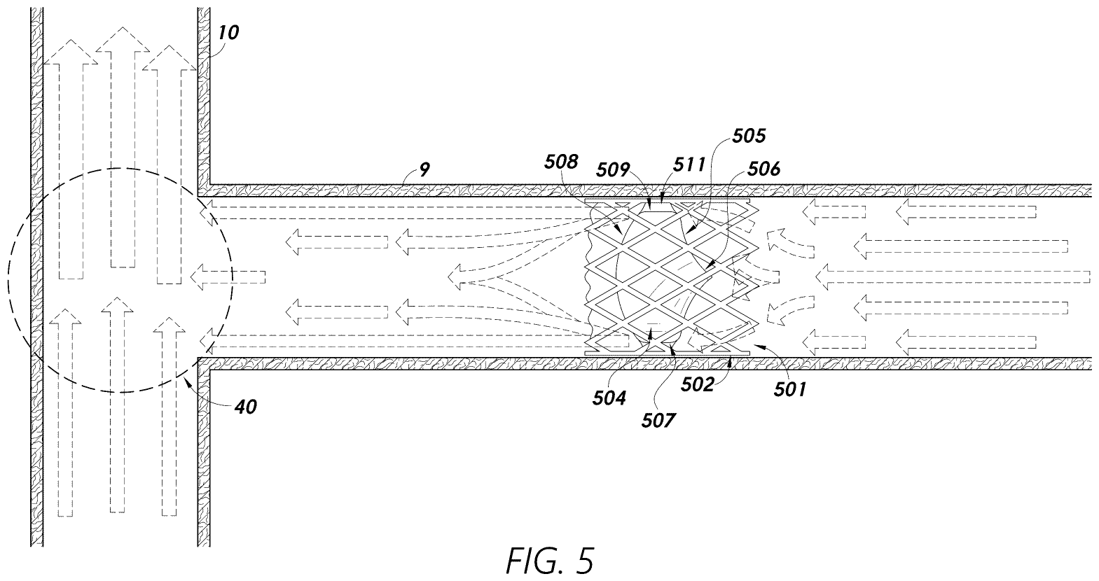

FIG. 5 illustrates an example medical implant 501 in a compressed form and disposed within a blood vessel 9 of a heart, in accordance with one or more examples. The medical implant 501 may comprise a stent body 502 and/or a valve 504. In some examples, the valve 504 may be similar to a prosthetic heart valve. For example, the valve 504 may comprise one or more leaflets and/or may form an opening for blood flow through the valve 504 and/or stent body 502. The opening of the valve 504 may face a direction of blood flow. For example, the opening may be formed at a distal end 506 of the valve 504 that is distal from a proximal end 508 of the valve 504. The distal end 506 may be disposed downstream of the proximal end 508.

The valve 504 may comprise a distal portion 505 configured to flatten and/or otherwise deform in response to increased blood pressure against an outer surface of the distal portion 505. As a result, the opening may reduce in size in response to increase blood pressure through the vessel 9. In some examples, the distal portion 505 may have a conical and/or tapered form with a decreasing diameter relative to a diameter of a proximal portion 507 and/or proximal end 508 of the valve 504 and/or to a diameter of the stent body 502. The distal portion 505 and/or distal end 506 may be configured to fully close in response to increase blood pressure such that the opening may not allow any blood flow. In some examples, the valve 504 may allow blood flow at sides 509 of the valve 504 when the opening is at least partially closed.

In some examples, the valve 504 may comprise sides 509 configured to contact and/or form a seal against the vessel 9. For example, the valve 504 may form a generally circular cross-section and/or may form a generally circular area of contact with the vessel 9. In some examples, the valve 504 may comprise one or more apertures 511 configured to allow blood flow through the valve 504 in the closed and/or compressed form shown in FIG. 5. In some examples, the apertures 511 may migrate towards a distal and/or flow-facing end of the valve 504 as the distal portion 505 closes. As the distal portion 505 flattens, the sides 509 may be pulled away from the walls of the vessel 9 and/or the valve 504 may form one or more gaps between the valve 504 and the walls of the vessel 9.

Increased blood pressure against the distal portion 505 may cause a reduction of a length of the valve 504. For example, the distal portion 505 may be pressed towards the proximal end 508 of the valve 504, reducing a distance between the distal end 506 and the proximal end 508 of the valve 504.

The valve 504 may comprise a covering extending at least partially along an inner surface and/or outer surface of an inner frame and/or skeleton (not shown) of the valve 504. The covering may comprise one or more fabrics, polymers, rubber, and/or other materials. In some examples, the covering may have a generally pliable, soft, and/or stretchy structure. The covering may be generally fluid-tight and/or may prevent blood flow through the covering. In some examples, the covering may comprise one or more bypass apertures configured to allow blood flow.

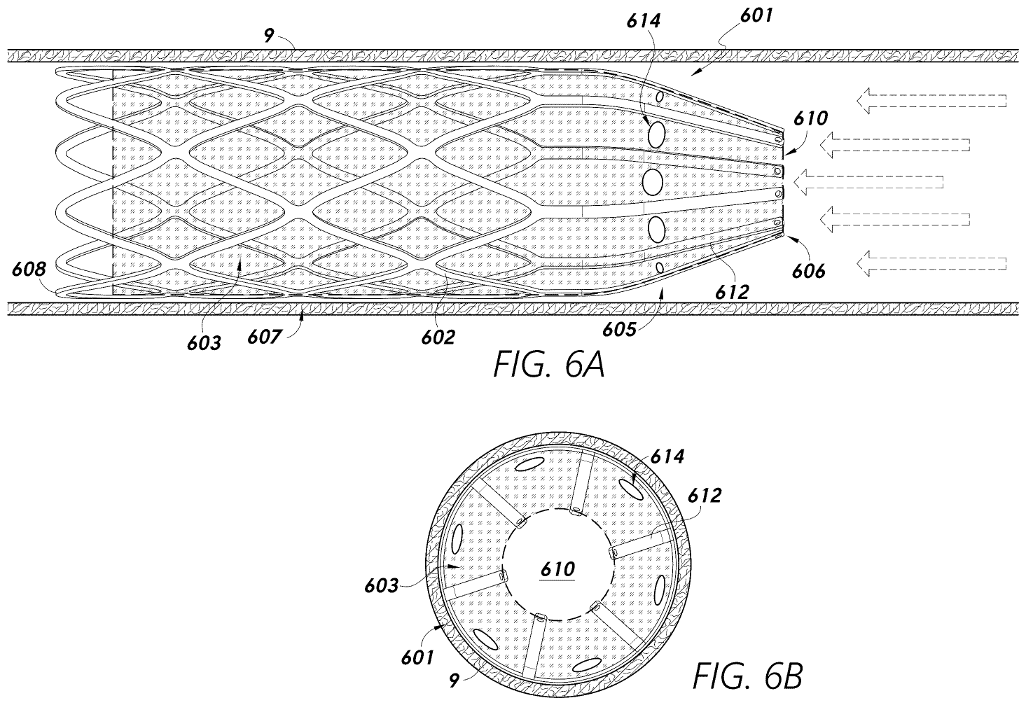

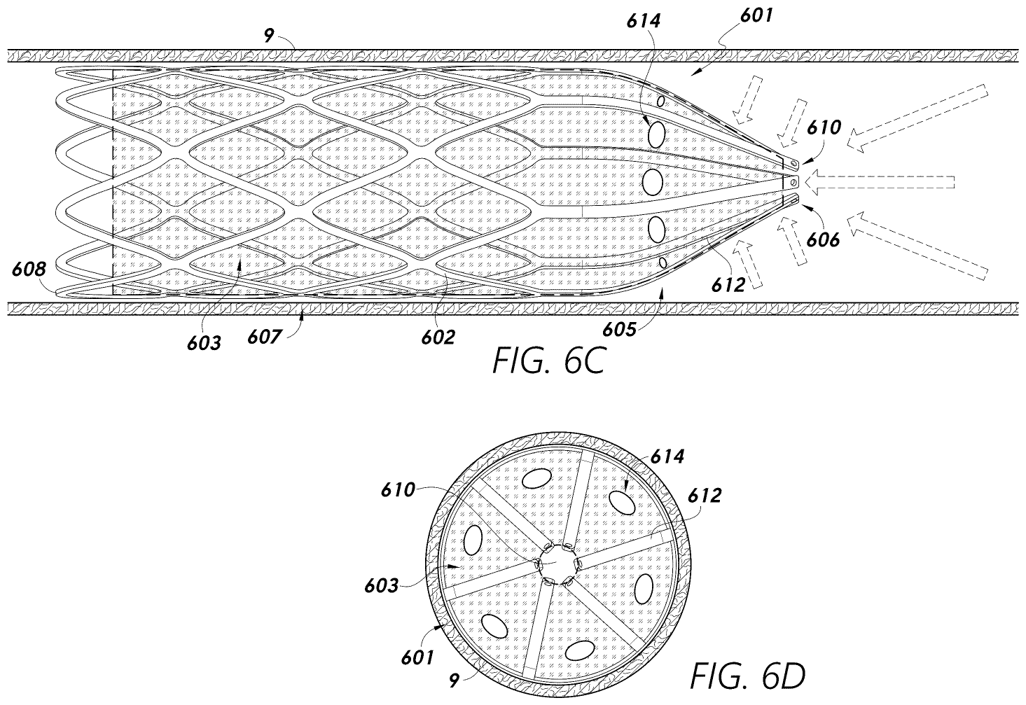

FIGS. 6A-6D illustrate an example occlusion device 601 (e.g., a prosthetic valve) disposed within a blood vessel 9 (e.g., a hepatic vein) in accordance with one or more examples. FIG. 6A provides a side view of the device 601 in an expanded and/or default form. The device 601 can comprise a stent body 602 (e.g., a cylindrical and/or tubular wire stent) and/or a covering 603 at least partially enclosing the stent body 602 and/or attached to an outer and/or inner surface of the stent body 602. In some examples, the covering 603 can be at least partially composed of cloth and/or other generally flexible materials. In some examples, the device 601 and/or stent body 602 may comprise a distal portion 605 having a generally conical (e.g., partial conical) and/or tapered shape and/or decreasing in diameter from a diameter of a proximal portion 607 of the stent body 602. The distal portion 605 can comprise one or more arms 612 and/or elongate members extending generally longitudinally and/or at an approximately 45-degree angle along the device 601. For example, the one or more arms 612 may be configured to extend towards a distal end 606 of the device 601 and/or generally opposite a direction of blood flow when the device 601 is placed within the vessel 9. The one or more arms 612 may be configured to extend towards each other and/or towards a common point (e.g., towards a central axis of the device 601). In some examples, the one or more arms 612 may be configured to bend (e.g., elastically bend) and/or may be at least partially bent in a default form to create a tapered form of the distal portion 605. The one or more arms 612 may be at least partially composed of Nitinol and/or other shape memory alloys configured to be shape set in the curved form shown in FIG. 6A. The covering 603 may be configured to extend along an outer surface and/or inner surface of the one or more arms 612 and/or may assume a generally conical and/or tapered shape at the distal portion 605 and/or may be configured to be shaped by the one or more arms 612.

While the device 601 is shown comprising six arms 612, the device 601 may comprise any number of arms 612. The arms 612 may be configured to extend at least partially towards each other and/or towards a common point (e.g., towards a central axis of the device 601). FIG. 6B provides a frontal view of the device 601 in the expanded and/or default form. As shown in FIG. 6B, the one or more arms 612 and/or covering 603 may be configured to form an opening 610 (e.g., orifice) about the central axis of the device 601. The opening 610 may be smaller than a maximal/maximum diameter of the stent body 602. In some examples, the one or more arms 612 may be at least partially offset and/or may not directly face and/or extend towards each other, as shown in FIG. 6B. In other examples, the arms 612 may comprise pairs of arms 612 situated and/or extending generally opposite each other across the opening 610.

In some examples, the proximal portion 607 of the stent body 602 may comprise a network of interconnected and/or interwoven wires and/or other elongate materials. The one or more arms 612 may extend from the proximal portion 607. As shown in FIG. 6A, the one or more arms 612 may be slightly closed (e.g., bent and/or angled) in a default and/or expanded form. The stent body 602 may comprise a proximal end 608 having a generally equal diameter relative to the proximal portion 607. The proximal end 608 may form an opening and/or orifice with a larger diameter than the distal end 606.

FIG. 6C provides a side view of the device 601 in an at least partially compressed form. As blood pressure and/or blood flow increases (e.g., during splanchnic volume redistribution), the distal portion 605 (e.g., the arms 612 and/or covering 603) may be configured to close. For example, blood flow may be directed against an outer surface of the covering 603 and/or arms 612 and/or may press against the covering 603 and/or arms 612 such that the distal portion 605 flattens and/or is pressed towards the stent body 602 and/or the opening 610 reduces in size. FIG. 6D provides a frontal view of the device 601 in the compressed form. Reduction of the size of the opening 610 reduces the amount of flow through the orifice of the device 601. In some examples, an angle of the one or more arms 612 relative to the proximal portion 607 may increase as the distal portion 605 flattens in response to increased pressure.

In some examples, the covering 603 can comprise one or more apertures 614 and/or bypass holes configured to receive blood flow and/or to allow blood flow into the orifice of the device 601. The one or more apertures 614 may be positioned at the distal portion 605 (e.g., at a generally curved portion of the covering 603) and/or between the proximal portion 607 and the distal portion 605 (e.g., at a transition portion between the covering 603 having a generally straight form at the proximal portion 607 and the covering 603 having a generally curved form at the distal portion 605. In some examples, the one or more apertures 614 may have generally oval (e.g., circular) shapes and/or may have any suitable shapes and/or sizes. The covering 603 may comprise at least one aperture 614 between each pair of arms 612 of the device 601. However, the covering 603 can alternatively comprise any number of apertures 614. In some examples, the apertures 614 can be configured to prevent and/or reduce stagnation and/or thrombus formation at or near the device 601.

While the device 601 is shown comprising a covering 603 and/or apertures 614, the device 601 and/or other devices herein may not comprise any covering 603 and/or apertures 614. For example, a device 601 may not comprise a generally solid stent body 602 and/or distal portion 605 and/or may comprise a generally dense network of wires such that the device 601 allows only limited blood flow through the stent body 602 and/or distal portion 605. The network of wires may form the tapered distal portion 605 and/or the opening 610 as described above.

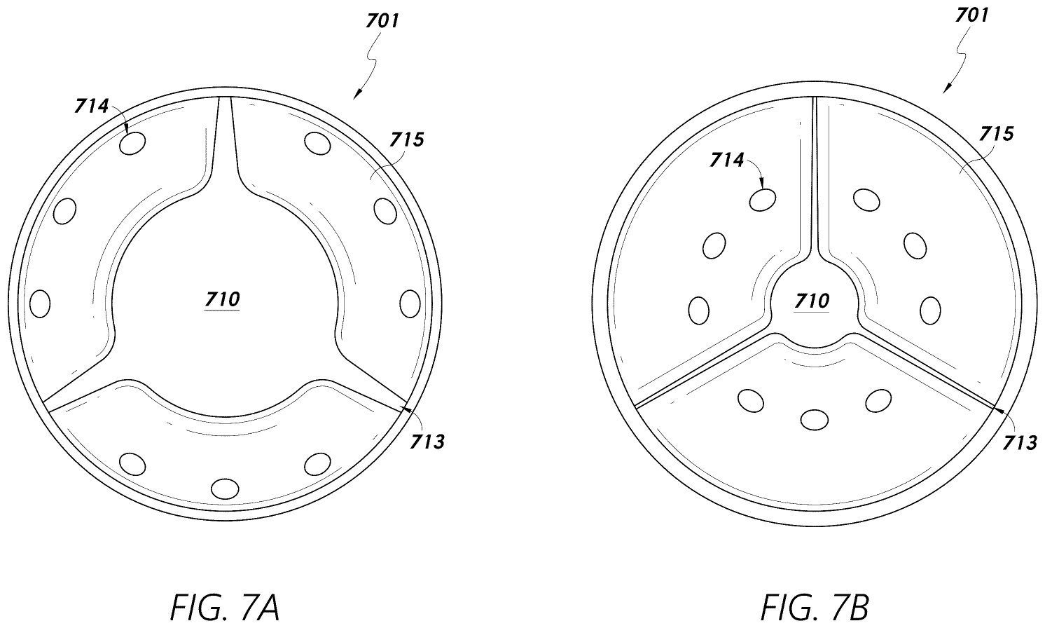

FIGS. 7A and 7B illustrate an example occlusion device 701 comprising one or more leaflets 715 and configured to be disposed within a blood vessel (e.g., a hepatic vein) in accordance with one or more examples. The term “leaflet” is used herein in accordance with its plain and ordinary meaning and may refer to any covering and/or appendage configured to at least partially extend over a lumen of an occlusion device (e.g., stent) and/or blood vessel. In some examples, the occlusion device 701 can comprise multiple leaflets 715 at least partially overlapping and/or extending in series around a circumference of the occlusion device 701. The one or more leaflets 715 can extend from an outer diameter of the occlusion device 701 towards a central axis of the occlusion device 701.

FIG. 7A provides a frontal view of the occlusion device 701 in an open and/or default state. In some cases, one or more of the leaflets 715 can comprise bypass apertures 714 (e.g., holes and/or openings) to allow blood flow through the leaflets 715 and/or to reduce thrombus formation. The bypass apertures 714 can be oriented along a generally circular path about a longitudinal axis and/or center of an opening 710 formed by the one or more leaflets 715. The apertures 714 can have any suitable size and/or shape. While the apertures 714 are shown having generally circular forms, example apertures 714 can comprise generally elongate and/or thin openings cut into one or more leaflets 715.

While the occlusion device 701 is shown with three leaflets 715, the occlusion device 701 can comprise any number of leaflets 715. In some examples, one or more leaflets 715 can extend from a covering configured to extend at least partially along a proximal portion (not shown; see, e.g., FIGS. 6A-6D) of the occlusion device 701. The one or more leaflets 715 can comprise a generally soft and/or flexible material configured to be at least partially responsive to pressure from blood passing through and/or around the occlusion device 701.

FIG. 7B provides a frontal view of the occlusion device 701 in an at least partially closed state. The occlusion device 701 may be disposed within a blood vessel such that blood flow through the vessel is directed towards the leaflets 715 of the device 701. For example, in the example shown in FIGS. 7A and 7B, blood flow may be directed into the page. As blood pressure increases, the increased blood flow may exert an increased amount of pressure on the leaflets 715 to cause the leaflets 715 to naturally close and/or cover the central opening 710 formed by the leaflets 715. Thus, the opening 710 may reduce in size as blood pressure increases, causing increased occlusion of blood flow. In some examples, the one or more leaflets 715 may form gaps 713 (e.g., laterally extending and/or elongate gaps 713) between the leaflets 715. These gaps 713 may similarly close in response to increased blood pressure. In some examples, flattening of the leaflets 715 in response to blood pressure may cause the one or more apertures 714 to move towards a central axis of the device 701, as shown in FIG. 7B. For example, the one or more apertures 714 may be disposed generally near edge portions of the one or more leaflets 715. Accordingly, as the edge portions migrate towards the central axis of the device 701 in response to flattening of the leaflets 715, the apertures 714 may similarly migrate towards the central axis.

The device 701 can comprise a stent body (not shown) and/or a covering at least partially enclosing the stent body. In some examples, the device 701 and/or leaflets 715 may form a generally conical and/or tapered shape and/or the device 701 may decrease in diameter from a maximal diameter of the stent body. The one or more leaflets 715 may be supported by one or more arms and/or elongate members extending generally longitudinally along the device 701. For example, the one or more leaflets 715 may extend along an outer surface of one or more elongate arms having a bent and/or tapered form.

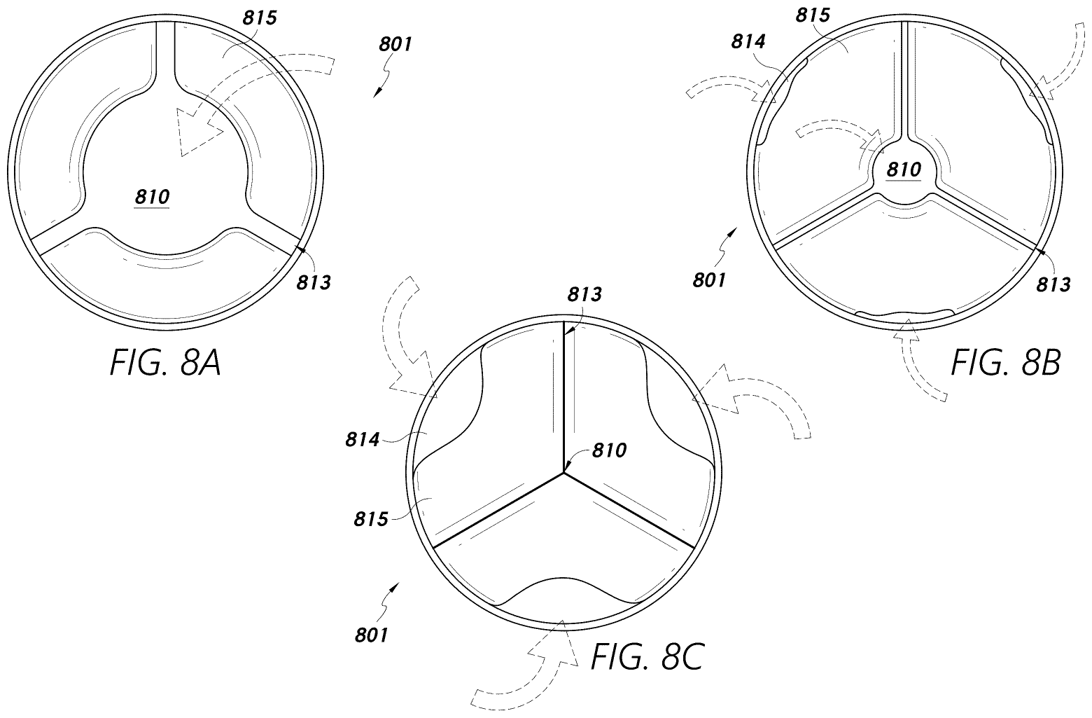

FIGS. 8A-8C illustrate another example occlusion device 801 comprising one or more leaflets 815 configured to be disposed within a blood vessel (e.g., a hepatic vein) in accordance with one or more examples. In some examples, the occlusion device 801 can comprise multiple leaflets 815 at least partially overlapping and/or extending in series around a circumference of the occlusion device 801.

FIG. 8A provides a frontal view of the occlusion device 801 in an open and/or default state. In some cases, one or more of the leaflets 815 can comprise bypass apertures 814 (e.g., holes) to allow blood flow through the leaflets 815 and/or to reduce thrombus formation. While the occlusion device 801 is shown with three leaflets 815, the occlusion device 801 can comprise any number of leaflets 815.

The bypass apertures 814 can be disposed at or near intersections points of the leaflets 815 and/or a stent body of the device 801. For example, the one or more apertures 814 may be disposed at a transition between a proximal portion of the stent body and a distal portion of the stent body. FIG. 8B provides a frontal view of the occlusion device 801 in an at least partially closed state and FIG. 8C provides a frontal view of the device 801 in a fully closed state. The occlusion device 801 may be disposed within a blood vessel such that blood flow through the vessel is directed towards the leaflets 815 of the device 801. For example, in the example shown in FIGS. 8A and 8B, blood flow may be directed into the page. As blood pressure increases, the increase blood flow may exert an increased amount of pressure on the leaflets 815 to cause the leaflets 815 to naturally close and/or cover the central opening 810 formed by the leaflets 815. Thus, the opening 810 may reduce in size as blood pressure increases, causing increased occlusion of blood flow. In some examples, the one or more leaflets 815 may form gaps 813 (e.g., laterally extending and/or elongate gaps 813) between the leaflets 815. These gaps 813 may similarly close in response to increased blood pressure.

The device 801 can comprise a stent body (not shown) and/or a covering at least partially enclosing the stent body. In some examples, the device 801 and/or leaflets 815 may form a generally conical and/or tapered shape and/or the device 801 may decrease in diameter from a diameter of stent body. The one or more leaflets 815 may be supported by one or more arms and/or elongate members extending generally longitudinally along the device 801.

The bypass apertures 814 may be configured to increase in size and/or to migrate towards a frontal area of the device 801 as blood pressure increases. For example, increased blood pressure may cause the one or more leaflets 815 to flatten and/or stretch across the opening 810 between the leaflets 815. As a result, the apertures 814 may extend further over a lumen of the device 801 and/or over a front-facing portion of the device 801. As a result, the apertures 814 may be drawn distally and/or towards a front-facing portion of the device 801 such that a front-facing portion/area of the apertures 814 increases and/or blood flow into the leaflets 815 may increasingly pass through the apertures 814 as blood pressure increases.

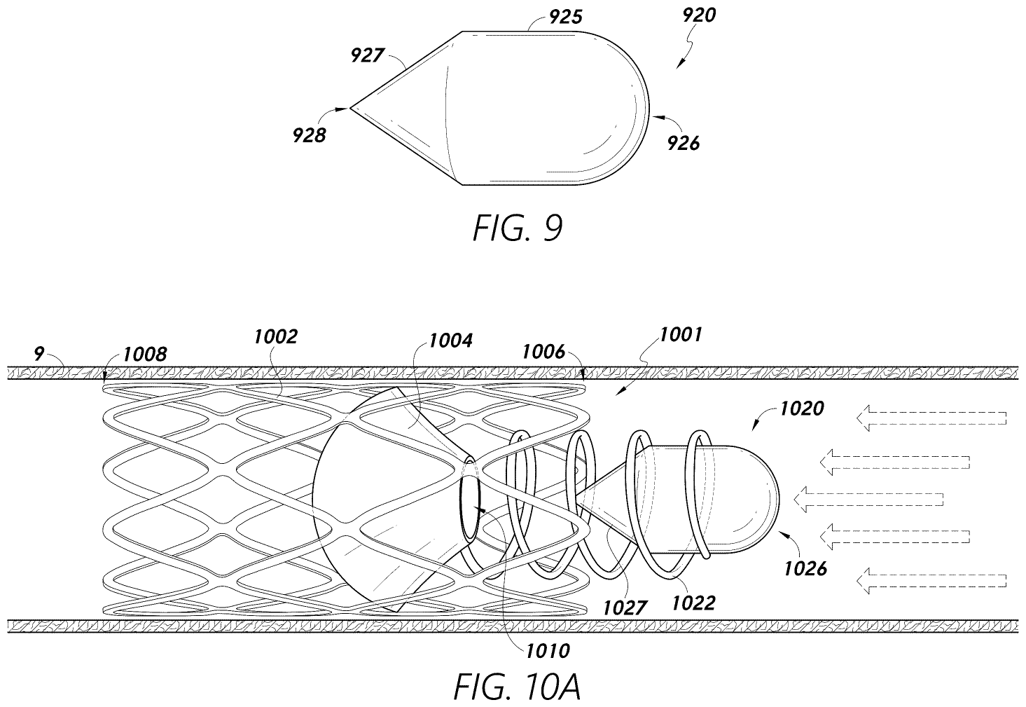

FIG. 9 illustrates an example plug 920 configured for plugging, blocking, and/or closing an opening of an occlusion device in accordance with one or more examples. In some examples, the plug 920 may comprise a wire and/or solid frame and/or similar material with a hollow and/or filled core. For example, the plug 920 may comprise a wire frame at least partially enclosed by a covering composed of fabric, polymer, and/or other materials. The plug 920 may comprise a proximal portion 927 and/or proximal end 928, a midsection 925, and/or a distal end 926. The proximal portion 927 may have a conical shape and/or may be configured to be extended at least partially into the occlusion device such that at least the proximal end 928 enters a lumen and/or opening of the occlusion device. The proximal portion 927 may be configured to fit into and/or mate with a generally oval shaped opening and/or orifice of the occlusion device.

The midsection 925 may have a generally cylindrical and/or tubular shape. The distal end 926 may be generally flat or rounded and/or may comprise a spherical cap and/or may have a semi-spherical and/or flat shape. In some examples, the distal end 926 may be configured to face a direction of blood flow and/or may have a suitable surface area such that blood flow against the distal end 926 applies a pushing force against the distal end 926.

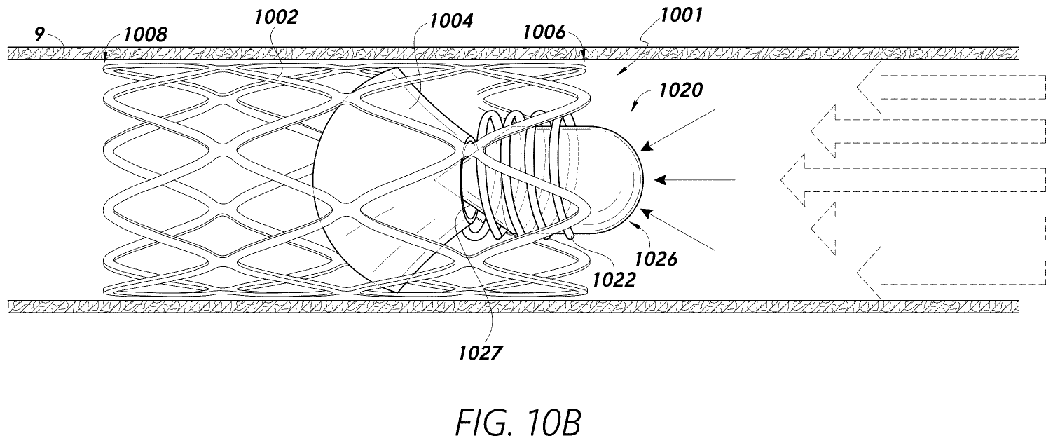

FIG. 10A illustrates an example occlusion device 1001 in a blood vessel 9 and comprising a plug 1020 in a default and/or expanded form in accordance with one or more examples. The plug 1020 may be coupled and/or attached to a distal end 1006 of a stent body 1002 of the occlusion device 1001. In some examples, the distal end 1006 of the stent body 1002 may comprise a generally oval shaped (e.g., circular) opening configured to at least partially receive the plug 1020. In some examples, the occlusion device 1001 may comprise a valve 1004 comprising an orifice configured to receive the plug 1020. The plug 1020 may comprise a proximal end 1027 configured to at least partially enter the opening 1010 at the distal end 1006 of the stent body 1002 and/or to enter an orifice of the valve 1004. The plug 1020 may increase in diameter from the proximal end 1027 to a midsection and/or distal end 1026 of the plug 1020. In some examples, the midsection may be wider than the opening 1010 at the distal end 1006 of the stent body 1002. Accordingly, the midsection may prevent the plug 1020 from fully passing through the opening 1010 and/or through the valve 1004 (i.e., restrictor valve). The valve 1004 may comprise a tapered distal portion comprising an orifice configured to receive the proximal end 1027 of the plug 1020. The stent body 1002 may have a generally cylindrical form extending between the distal end 1006 and a proximal end 1008.

In some examples, a tether 1022 may extend from the stent body 1002 and/or may couple to the plug 1020. For example, the tether 1022 may comprise a coil, spring, wire, string, cord, tether, and/or similar device. In some examples, the tether 1022 may at least partially encircle the plug 1020 and/or form one or more coils around the plug 1020. The tether 1022 may be configured to bias the plug 1020 to be spaced at least partially away from and/or distally from the stent body 1002 to form a gap between the plug 1020 and the stent body 1002. For example, the tether 1022 may have a generally solid form and/or may have sufficient rigidity to hold the plug 1020 away from the stent body 1002 in the absence of external forces. Accordingly, blood may be able to pass through a gap between the plug 1020 and the opening of the valve 1004 and/or into an orifice of the stent body 1002 in the default and/or biased form.

FIG. 10B illustrates the example occlusion device 1001 in a compressed form in accordance with one or more examples. In some examples, an internal bias of the tether 1022 may be configured to be overcome by pressure from increased blood flow through the vessel 9. In response to increased blood pressure, the plug 1020 may be configured to be pulled towards the opening 1010 of the stent body 1002 to at least partially close the opening 1010 and/or prevent blood flow into the opening 1010. Increases in blood pressure at or near the device 1001 can cause movement of the stent body 1002 to naturally press the tether 1022 and/or plug 1020 towards the opening 1010. Similarly, decreases in blood pressure can cause relaxation of the device 1001 and/or movement of the plug 1020 and/or tether 1022 away from the opening 1010.

While the tether 1022 is shown having a helical coil form, the tether 1022 may comprise other forms. For example, the tether 1022 may comprise one or more beam springs and/or braided wires.

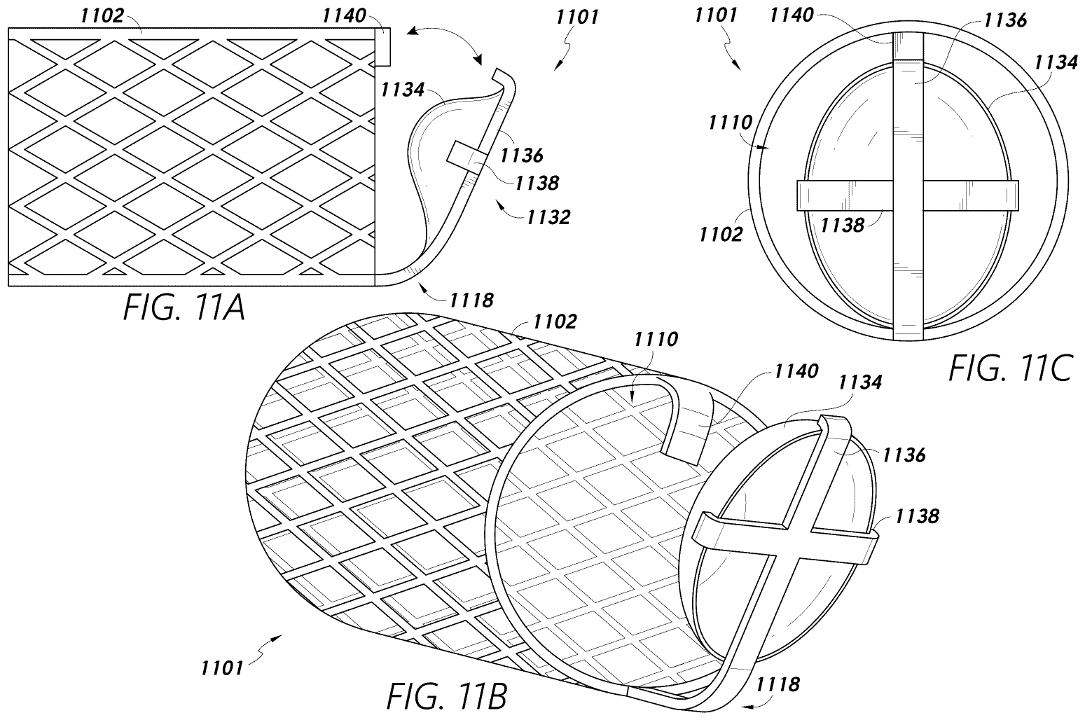

FIGS. 11A-11C illustrate another example occlusion device 1101 configured to selectively occlude a blood vessel in accordance with one or more examples. FIG. 11A provides a side view of the device 1101 in a relaxed and/or default form, FIG. 11B provides a perspective view of the device 1101 in the relaxed and/or default form, and FIG. 11C provides a frontal view of the device 1101 in a compressed form.

The device 1101 may comprise a stent body 1102 and/or a valve coupled to the stent body 1102 via a tether 1118. The valve may comprise a frame 1132 and/or a cap 1134. The stent body 1102 may comprise a stopper 1140 configured to prevent the frame 1132 from entering an orifice 1110 of the stent body 1102. The cap 1134 may be configured to enter at least partially into the orifice 1110 of the stent body 1102. The stopper 1140 may be configured to extend at least partially over the orifice 1110 and/or inner lumen of the stent body. In some examples, the stopper 1140 may have a generally rectangular form and/or may have any suitable size and/or shape.

The cap 1134 may be at least partially composed of silicone and/or other generally solid materials. The cap 1134 may be configured to block blood flow. In some examples, the cap 1134 may have a semispherical and/or bowl-shaped form. For example, the cap 1134 may extend away from a midpoint of the frame 1132. In some examples, the cap 1134 may be oval-shaped and/or may have a greater length than width. The length of the cap 1134 may be approximately equal to a diameter of the stent body 1102 and/or the width of the cap 1134 may be less than the diameter of the stent body 1102.

The frame 1132 may comprise crossing arms, including a first arm 1136 and/or a second arm 1138. The first arm 1136 may extend over a length of the cap 1134 and/or the second arm 1138 may extend over a width of the cap 1134. The first arm 1136 and the second arm 1138 may intersect at a midpoint of the cap 1134. In some examples, the second arm 1138 may dip down and/or extend away from the first arm 1136. For example, the first arm 1136 may approximate a curvature of the cap 1134.

In some examples, the cap 1134 may have a concave form relative to a direction of blood flow. For example, when positioned in a blood vessel, blood may flow into a bowl-shaped interior of the cap 1134. The tether 1118 may be at least partially rigid and/or may be configured to at least partially resist movement due to pressure applied to relatively low blood pressure. However, in response to increased blood pressure, the rigidity of the tether 1118 may be overcome and/or the cap 1134 may be pressed towards the orifice 1110 of the stent body 1102.

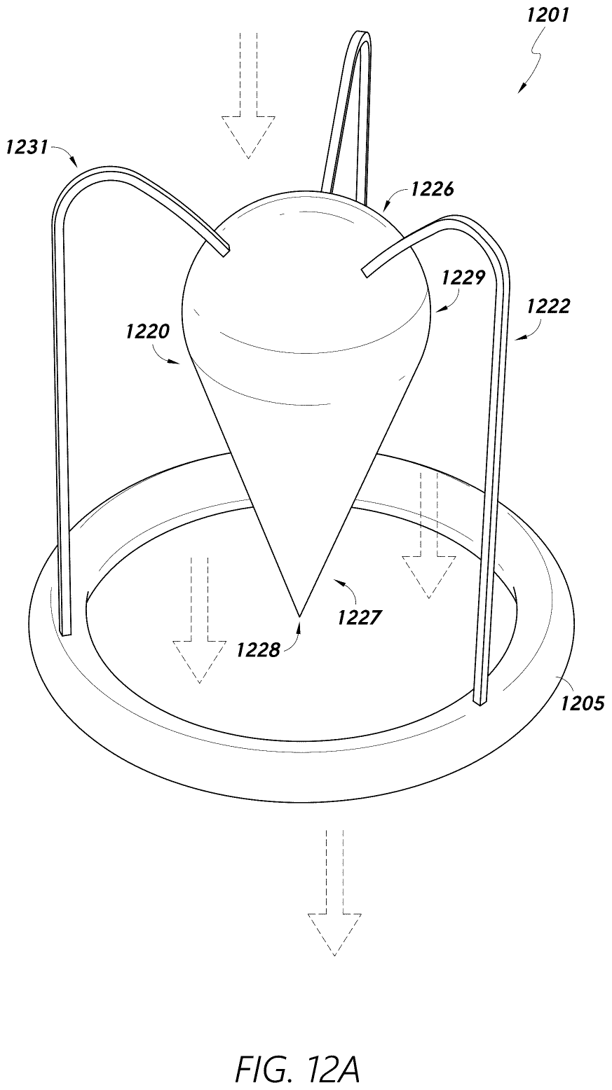

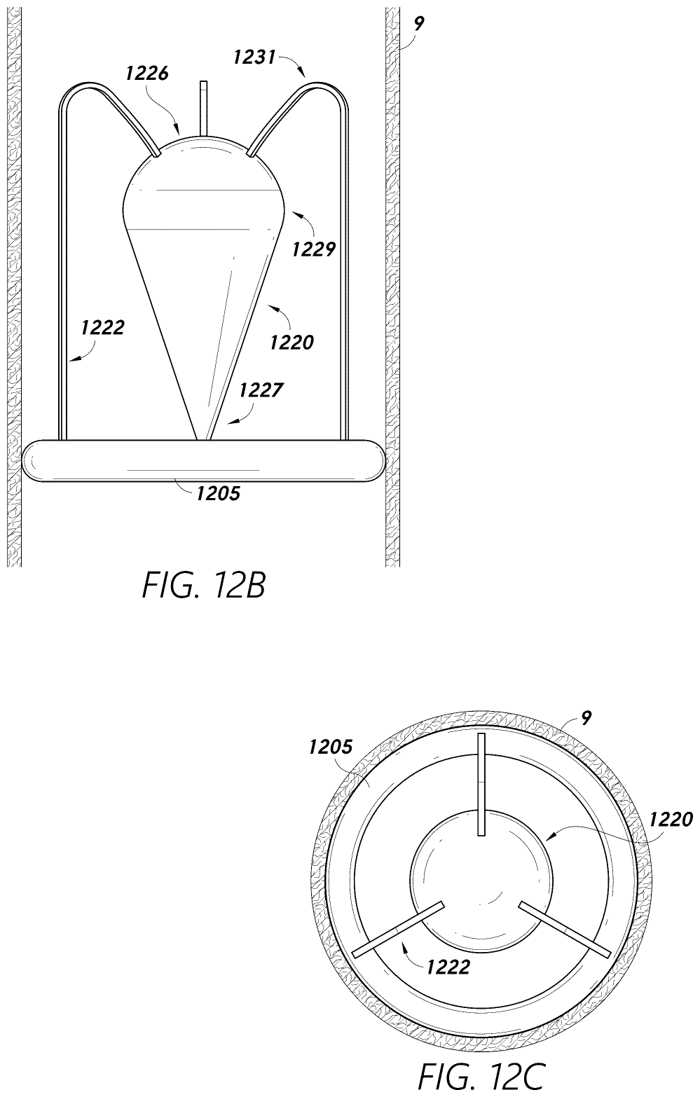

FIGS. 12A-12C illustrates an example occlusion device 1201 in a blood vessel 9 and comprising a plug 1220 in a default and/or expanded form in accordance with one or more examples. FIG. 12A provides a perspective view of the occlusion device 1201, FIG. 12B provides a side view of the occlusion device 1201, and FIG. 12C provides a top view of the occlusion device 1201. An example plug 1220 may be configured for plugging, blocking, and/or closing an opening of a mounting ring 1205 of the occlusion device 1201 in accordance with one or more examples. In some examples, the plug 1220 may comprise a wire and/or solid frame and/or similar material with a hollow and/or filled core. For example, the plug 1220 may comprise a wire frame at least partially enclosed by a covering composed of fabric, polymer, and/or other materials. The plug 1220 may comprise a proximal portion 1227 and/or proximal end 1228, a midsection 1229, and/or a distal end 1226. The proximal portion 1227 may have a conical shape and/or may be configured to be extended at least partially into the mounting ring 1205 such that at least the proximal end 1228 enters a lumen and/or opening of the mounting ring 1205. The proximal portion 1227 may be configured to fit into and/or mate with a generally oval shaped opening and/or orifice of the mounting ring 1205.

The midsection 1229 may have a generally cylindrical and/or tubular shape and/or may comprise a greatest diameter and/or width of the plug 1220. The distal end 1226 may be generally flat or rounded and/or may comprise a spherical cap and/or may have a semi-spherical and/or flat shape. In some examples, the distal end 1226 may be configured to face a direction of blood flow and/or may have a suitable surface area such that blood flow against the distal end 1226 applies a pushing force against the distal end 1226.

The plug 1220 may be coupled and/or attached to a mounting ring 1205, stent, and/or other anchoring device. In some examples, mounting ring 1205 may comprise a generally oval shaped (e.g., circular) opening configured to at least partially receive the plug 1220. In some examples, the mounting ring 1205 may comprise a valve and/or an orifice configured to receive the plug 1220. The plug 1220 may comprise a proximal end 1228 configured to at least partially enter the opening of the mounting ring 1205 and/or a valve of the mounting ring 1205. The plug 1220 may increase in diameter from the proximal end 1228 to a midsection and/or distal end 1226 of the plug 1220. In some examples, the midsection 1229 may be wider than the opening of the mounting ring 1205. Accordingly, the midsection 1229 may prevent the plug 1220 from fully passing through the opening of the mounting ring 1205 and/or through the valve (e.g., restrictor valve). However, the opening of the mounting ring 1205 may be wider than the midsection 1229 of the plug 1220

In some examples, the plug 1220 may be coupled to the mounting ring 1205 via one or more arms 1222. The one or more arms 1222 may be configured to provide a movable coupling between the plug 1220 and the mounting ring 1205. In some examples, the one or more arms may be at least partially flexible and/or bendable. Each of the one or more arms 1222 may comprise a bend 1231 and/or may couple to the mounting ring 1205 and/or to the distal end 1226 of the plug 1220. In response to blood pressure against the distal end 1226, the bend 1231 of the one or more arms 1222 may migrate along the one or more arms 1222 as the plug 1220 moves towards the mounting ring 1205. The one or more arms 1222 may be configured to allow and/or enable movement of the plug 1220 relative to the mounting ring 1205. For examples, the mounting ring 1205 may remain anchored and/or otherwise in a constant position relative to surrounding tissue and/or the plug 1220 may be configured to move relative to the mounting ring 1205 (e.g., towards the mounting ring 1205 and/or away from the mounting ring 1205).

The one or more arms 1222 may comprise a coil, spring, wire, string, cord, tether, and/or similar device. The occlusion device 1201 may comprise any number of arms 1222. While three arms 1222 are shown in FIGS. 12A-12C. In some examples, the one or more arms 1222 may be generally evenly spaced around the mounting ring 1205 and/or around the plug 1220. The one or more arms 1222 may be configured to bias the plug 1220 to be spaced at least partially away from and/or distally from the mounting ring 1205 to form a gap between the plug 1220 and/or at least the midsection 1229 of the plug 1220 and the mounting ring 1205. For example, the one or more arms 1222 may have a generally solid form and/or may have sufficient rigidity to hold the plug 1220 away from the mounting ring 1205 in the absence of external forces. Accordingly, blood may be able to pass through a gap between the plug 1220 and the opening of the mounting ring 1205 and/or into an orifice of the mounting ring 1205 in the default and/or biased form.

In some examples, an internal bias of the one or more arms 1222 may be configured to be overcome by pressure from increased blood flow through the vessel 9. In response to increased blood pressure, the plug 1220 may be configured to be pulled towards the mounting ring 1205 to at least partially close the opening of the mounting ring 1205 and/or prevent blood flow into the mounting ring 1205. Increases in blood pressure at or near the device 1201 can cause movement of the plug 1220 towards the mounting ring 1205. Similarly, decreases in blood pressure can cause relaxation of the device 1201 and/or movement of the plug 1220 away from the mounting ring 1205.

The plug 1220 may have a generally conical shape and/or may gradually decrease in diameter and/or width from the midsection 1229 to the proximal end 1228. As the plug 1220 gradually descends into the mounting ring 1205, blockage of blood flow through the mounting ring 1205 may gradually increase from the proximal end 1228 to the midsection 1229 due to the gradually increasing diameter and/or width of the plug 1220.

In some examples, the mounting ring 1205 and/or arms 1222 may be generally flexible and/or may be configured to compress to allow for transcatheter delivery via one or more catheters and/or shafts. For example, the arms 1222 and/or mounting ring 1205 may be configured to collapse around the plug 1220. The plug 1220 may be sized to fit within one or more catheters without requiring compression of the plug 1220. However, the plug 1220 may be at least partially compressible. In some examples, the plug 1220 may comprise a rigid and/or braided structure. For example, the plug 1220 may comprise a network of braided polyurethane lines and/or strips configured to form the plug 1220. In some examples, the plug 1220 may be at least partially hollow within an outer structure.

The one or more arms 1222 and/or mounting ring 1205 may be at least partially composed of one or more shape-memory alloys (e.g., Nitinol). In some examples, the one or more arms 1222 and/or mounting ring 1205 may be shape-set in a desired form (e.g., in the form shown in FIG. 12A) prior to delivery into a patient's body. Accordingly, the device 1201 may be configured to naturally assume the form shown in FIG. 12A in the absence of substantially high pressure against the plug 1220 and/or device 1201. The one or more arms 1222 may be configured to elastically deform in response to blood pressure against and/or or around the plug 1220 (e.g., against the distal end 1226 of the plug 1220). The proximal end 1228 of the plug 1220 may be disposed proximally relative to the mounting ring 1205 in the default state of the one or more arms 1222 and/or device 1201. In some examples, the one or more 1222 may be configured to allow the plug to move into and/or through the mounting ring 1205 such that the distal end 1226 of the plug 1220 may be disposed distally relative to the mounting ring 1205.

In some examples, the one or more arms 1222 may have varying width and/or density. For example, rigidity of the one or more arms 1222 may increase towards a connection point with the mounting ring 1205 based at least in part on gradually changing width and/or density of the one or more arms 1222. Accordingly, the one or more arms 1222 may be more susceptible to bending at or near connection points with the plug 1220 than at or near connection points with the mounting ring 1205.

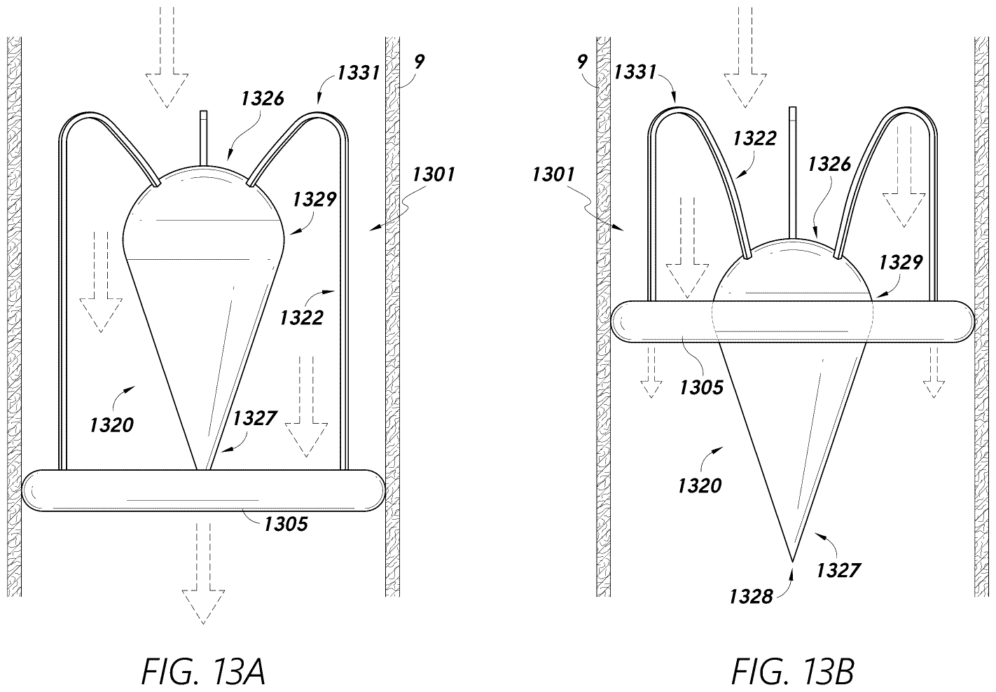

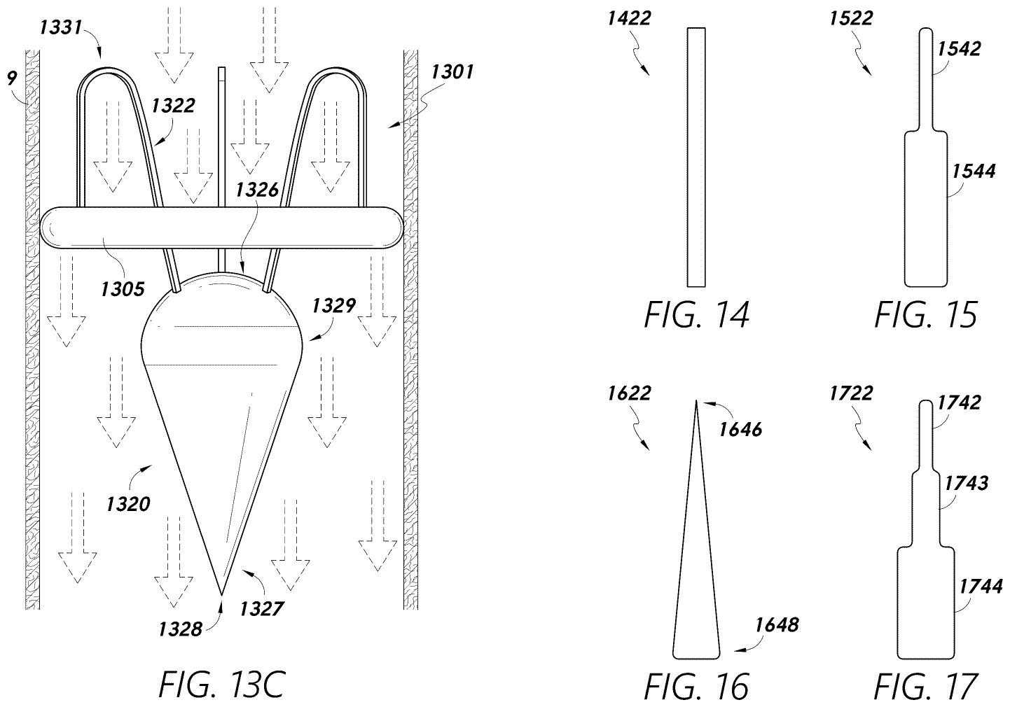

FIGS. 13A-13C illustrates an example occlusion device 1301 in a blood vessel 9 and comprising a plug 1320 in accordance with one or more examples. FIG. 13A provides a side view of the occlusion device 1301 in a default and/or expanded form, FIG. 13B provides a side view of the occlusion device 1301 in a closed and/or partially deformed state, and FIG. 13C provides a side view of the occlusion device 1301 in an open and/or fully deformed state. An example plug 1320 may be configured for plugging, blocking, and/or closing an opening of a mounting ring 1305 of the occlusion device 1301 in accordance with one or more examples. In some examples, the plug 1320 may comprise a wire and/or solid frame and/or similar material with a hollow and/or filled core. For example, the plug 1320 may comprise a wire frame at least partially enclosed by a covering composed of fabric, polymer, and/or other materials. The plug 1320 may comprise a proximal portion 1327 and/or proximal end 1328, a midsection 1329, and/or a distal end 1326. The proximal portion 1327 may have a conical shape and/or may be configured to be extended at least partially into the mounting ring 1305 such that at least the proximal end 1328 enters a lumen and/or opening of the mounting ring 1305. The proximal portion 1327 may be configured to fit into and/or mate with a generally oval shaped opening and/or orifice of the mounting ring 1305.

The midsection 1329 may have a generally cylindrical and/or tubular shape and/or may comprise a greatest diameter and/or width of the plug 1320. The distal end 1326 may be generally flat or rounded and/or may comprise a spherical cap and/or may have a semi-spherical and/or flat shape. In some examples, the distal end 1326 may be configured to face a direction of blood flow and/or may have a suitable surface area such that blood flow against the distal end 1326 applies a pushing force against the distal end 1326.

The plug 1320 may be coupled and/or attached to a mounting ring 1305, stent, and/or other anchoring device. In some examples, mounting ring 1305 may comprise a generally oval shaped (e.g., circular) opening configured to at least partially receive the plug 1320. In some examples, the mounting ring 1305 may comprise a valve comprising an orifice configured to receive the plug 1320. The plug 1320 may comprise a proximal end 1328 configured to at least partially enter the opening at the mounting ring 1305 and/or a valve of the mounting ring 1305. The plug 1320 may increase in diameter from the proximal end 1328 to a midsection and/or distal end 1326 of the plug 1320. In some examples, the midsection 1329 may be wider than the opening of the mounting ring 1305. Accordingly, the midsection 1329 may prevent the plug 1320 from fully passing through the opening of the mounting ring 1305 and/or through the valve (e.g., restrictor valve). However, the opening of the mounting ring 1305 may be wider than the midsection 1329 of the plug 1320

In some examples, the plug 1320 may be coupled to the mounting ring 1305 via one or more arms 1322. The one or more arms 1322 may be configured to provide a movable coupling between the plug 1320 and the mounting ring 1305. In some examples, the one or more arms may be at least partially flexible and/or bendable. Each of the one or more arms 1322 may comprise a bend 1331 and/or may couple to the mounting ring 1305 and/or to the distal end 1326 of the plug 1320. In response to blood pressure against the distal end 1326, the bend 1331 of the one or more arms 1322 may migrate along the one or more arms 1322 as the plug 1320 moves towards the mounting ring 1305.

The one or more arms 1322 may comprise a coil, spring, wire, string, cord, tether, and/or similar device. The occlusion device 1301 may comprise any number of arms 1322. While three arms 1322 are shown in FIGS. 13A-12C. In some examples, the one or more arms 1322 may be generally evenly spaced around the mounting ring 1305 and/or around the plug 1320. The one or more arms 1322 may be configured to bias the plug 1320 to be spaced at least partially away from and/or distally from the mounting ring 1305 to form a gap between the plug 1320 and/or at least the midsection 1329 of the plug 1320 and the mounting ring 1305. For example, the one or more arms 1322 may have a generally solid form and/or may have sufficient rigidity to hold the plug 1320 away from the mounting ring 1305 in the absence of external forces. Accordingly, blood may be able to pass through a gap between the plug 1320 and the opening of the mounting ring 1305 and/or into an orifice of the mounting ring 1305 in the default and/or biased form.

In some examples, an internal bias of the one or more arms 1322 may be configured to be overcome by pressure from increased blood flow through the vessel 9. As shown in FIG. 13B, in response to increased blood pressure, the plug 1320 may be configured to be pulled towards the mounting ring 1305 and/or in the direction of the flow of blood to at least partially close the opening of the mounting ring 1305 and/or prevent blood flow into the mounting ring 1305. Increases in blood pressure at or near the device 1301 can cause movement of the plug 1320 towards the mounting ring 1305. Similarly, decreases in blood pressure can cause relaxation of the device 1301 and/or movement of the plug 1320 away from the mounting ring 1305. As the plug 1320 is moved in the direction of the flow of blood, the portion of the plug 1320 situated within the mounting ring 1305 may be gradually wider and wider until the midsection 1329 of the plug 1320 is situated within the mounting ring 1305 (closed and/or partially deformed state), based at least in part on a conical and/or tapered shape of the plug 1320. With the midsection 1329 disposed within the mounting ring 1305, the occlusion device 1301 may be in a closed state and/or may allow minimal blood flow through the mounting ring 1305.

If the plug 1320 continues to be pushed in the direction of blood flow after reaching the closed form shown in FIG. 13B, the one or more arms 1322 may continue to bend to allow advancement of the plug 1320 below the mounting ring 1305. As the plug 1320 continues to move downstream, the portion of the plug 1320 situated within the mounting ring 1305 may be gradually thinner and thinner beyond the midsection 1329 based at least in part on a conical and/or rounded form of the distal end 1326 of the plug 1320. As shown in FIG. 13C, the plug 1320 may eventually be pushed fully beyond the mounting ring 1305, resulting in an open state of the occlusion device 1301 and/or maximal blood flow through the mounting ring 1305.

FIGS. 14-17 illustrate various example arms for interconnecting an example plug to an example mounting ring. While each of FIGS. 14-17 illustrate a single arm, an occlusion device can comprise multiple arms (see, e.g., FIGS. 12A-12C). Each of the arms of an occlusion device can have a common structure and/or form and/or can have different structures and/or forms.

FIG. 14 illustrates an arm 1422 having a generally constant width. The arm 1422 have a tubular, rectangular, octagonal, and/or similar form.

FIG. 15 illustrates an arm 1522 having a single-tiered variable width. The arm 1522 may comprise a proximal portion 1544 and/or a distal portion 1542. The distal portion 1542 may have a smaller width than the proximal portion 1544. The arm 1522 may have a step-like width and/or form between the proximal portion 1544 and the distal portion 1542. In some examples, the arm 1522 may comprise a single tier and/or single step-like width change between the proximal portion 1544 and the distal portion 1542.

FIG. 16 illustrates an arm 1622 having a gradually-changing and/or variable width. The arm 1622 may comprise a proximal end 1648 and/or a distal end 1646. The distal end 1646 may have a smaller width than the proximal end 1648. The arm 1622 may have a tapered and/or conical form and/or may decrease gradually in width between the proximal end 1648 and the distal end 1646.

FIG. 17 illustrates an arm 1722 having a multi-tiered variable width. The arm 1722 may comprise a proximal portion 1744, a middle portion 1743, and/or a distal portion 1742. The distal portion 1742 may have a smaller width than the middle portion 1743 and/or the middle portion 1743 may have a smaller width than the proximal portion 1744. The arm 1722 may have a step-like form between the proximal portion 1744 and the middle portion 1743 and/or between the middle portion 1743 and the distal portion 1742. In some examples, the arm 1722 may comprise multiple (e.g., two) tiers and/or multiple (e.g., two) step-like width changes between the proximal portion 1744 and the distal portion 1742.

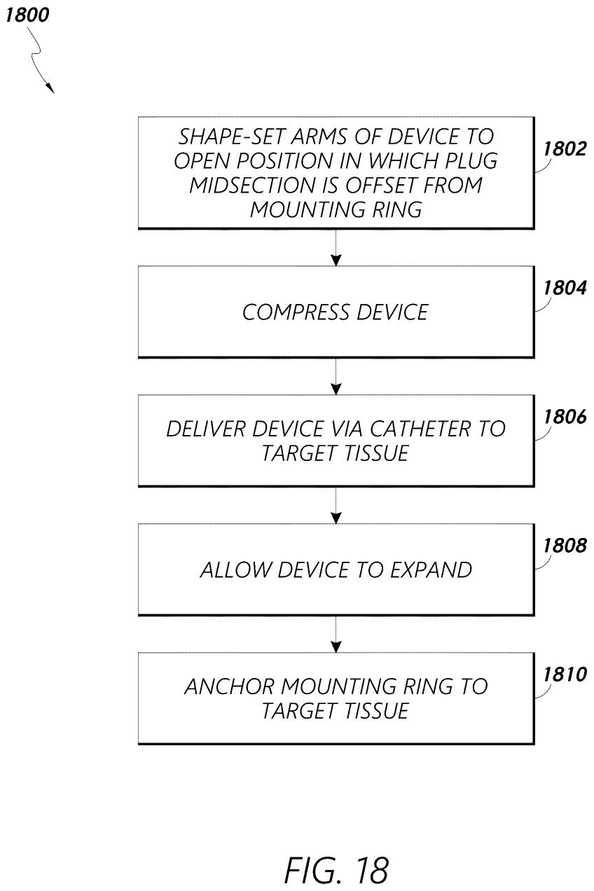

FIG. 18 provides a flowchart illustrating an example process 1800 of delivering and/or anchoring the various occlusion devices described herein. Steps of the process 1800 may be performed in any suitable order and/or steps may be removed and/or added as needed.

At step 1802, the process 1800 involves shape-setting one or more arms of an occlusion device to an open position in which at least a midsection of the plug is at least partially offset from the mounting ring. The one or more arms may interconnect the plug and the mounting ring and/or may be at least partially flexible. In some examples, the one or more arms may be at least partially composed of Nitinol and/or other shape memory alloys.

The midsection may comprise a portion of largest diameter and/or width of the plug. In some examples, the plug may be fully offset from the mounting ring in the open position and/or state. For examples, the plug may be disposed upstream of the mounting ring when the occlusion device is delivered to a blood vessel.

At step 1804, the process 1800 involves compressing the occlusion device. In some examples, the one or more arms and/or mounting ring may be at least partially flexible to allow for bending and/or compression. The one or more arms and/or mounting ring may be at least partially composed of one or more shape memory alloys.

At step 1806, the process 1800 involves delivering the occlusion device via a catheter to a target tissue site (e.g., an interior of a blood vessel). The occlusion device may be compressible to fit into any suitable catheter.

At step 1808, the process 1800 involves removing the occlusion device from the catheter and/or allowing the occlusion device to expand. In some examples, the occlusion device may be configured to naturally expand in response to removal from the catheter. For example, the one or more arms and/or mounting ring may return to unbent and/or shape-set forms following removal from the catheter.

At step 1810, the process 1800 involves anchoring the mounting ring to the target tissue (e.g., to interior walls of the blood vessel). In some examples, the mounting ring may be configured to anchor via friction between sides of the mounting ring and the interior walls of the blood vessel due to outward expansion of the mounting ring. However, various anchoring features may be used to facilitate anchoring of the mounting ring. For example, the mounting ring may comprise one or more fingers, needles, screws, hooks, and/or similar features configured to pierce and/or embed into the native tissue.

Described herein are various example medical implants and/or delivery methods. Some examples described herein may be used in combination and/or may be used independently.

Example 1: A medical implant for managing blood flow through a blood vessel, the medical implant comprising a stent body having an inner lumen and a restrictor valve having a tapered distal end extending at least partially over the inner lumen of the stent body.

Example 2: The medical implant of any example herein, wherein the tapered distal end of the restrictor valve is configured to face a direction of blood flow through the blood vessel.

Example 3: The medical implant of any example herein, wherein the tapered distal end of the restrictor valve is configured to at least partially flatten in response to increase blood pressure through the blood vessel.

Example 4: The medical implant of any example herein, wherein the blood vessel is an inferior vena cava.

Example 5: The medical implant of any example herein, wherein the tapered distal end of the restrictor valve forms an orifice into the inner lumen of the stent body.

Example 6: The medical implant of any example herein, wherein the orifice is at a central position of the restrictor valve.

Example 7: The medical implant of any example herein, wherein the tapered distal end of the restrictor valve is configured to reduce a size of the orifice in response to increased blood pressure through the blood vessel.

Example 8: The medical implant of any example herein, wherein the tapered distal end of the restrictor valve is configured to fully close the orifice in response to increased blood pressure through the blood vessel.

Example 9: The medical implant of any example herein, wherein the restrictor valve comprises one or more bypass apertures to allow blood flow through the restrictor valve.

Example 10: The medical implant of any example herein, wherein the one or more bypass apertures are disposed at the tapered distal end of the restrictor valve.

Example 11: The medical implant of any example herein, wherein the one or more bypass apertures are disposed at a proximal portion of the restrictor valve.

Example 12: The medical implant of any example herein, wherein the one or more bypass apertures are disposed at a transition between a proximal portion of the restrictor valve and the tapered distal end of the restrictor valve.

Example 13: The medical implant of any example herein, wherein the one or more bypass apertures are configured to increase in size in response to flattening of the tapered distal end of the restrictor valve.

Example 14: The medical implant of any example herein, wherein the tapered distal end of the restrictor valve comprises two or more leaflets.

Example 15: The medical implant of any example herein, wherein the two or more leaflets at least partially overlap.

Example 16: The medical implant of any example herein, wherein the restrictor valve comprises a covering extending at least partially over an outer surface of the stent body.

Example 17: The medical implant of any example herein, wherein the restrictor valve comprises a covering extending at least partially along an inner surface of the stent body.

Example 18: The medical implant of any example herein, wherein the stent body comprises one or more curved arms configured to support the tapered distal end of the restrictor valve.

Example 19: The medical implant of any example herein, further comprising a plug coupled to the stent body via a tether.

Example 20: The medical implant of any example herein, wherein the tether comprises a coiled wire.

Example 21: The medical implant of any example herein, wherein the tether is configured to hold the plug distally from the stent body.

Example 22: The medical implant of any example herein, wherein the tether is configured to allow the plug to move towards the stent body in response to increase blood pressure through the blood vessel.

Example 23: The medical implant of any example herein, wherein the plug is configured to fit into an orifice at the tapered distal end of the restrictor valve.

Example 24: The medical implant of any example herein, wherein the plug comprises a conical proximal end.

Example 25: The medical implant of any example herein, wherein the plug comprises a rounded distal end.

Example 26: The medical implant of any example herein, wherein the restrictor valve is coupled to the stent body via a tether.

Example 27: The medical implant of any example herein, wherein the restrictor valve comprises a bowl forming a spherical cap extending towards the inner lumen of the stent body and a concave interior facing a direction of blood flow through the blood vessel.

Example 28: The medical implant of any example herein, wherein the restrictor valve comprises crossing arms supporting the bowl.

Example 29: The medical implant of any example herein, wherein the stent body comprises a stopper configured to prevent at least a portion of the crossing arms from entering the inner lumen of the stent body.

Example 30: A method comprising percutaneously delivering, via a catheter, a medical implant for managing blood flow through a blood vessel, the medical implant comprising a stent body having an inner lumen and a restrictor valve having a tapered distal end extending at least partially over the inner lumen of the stent body.

### Additional Examples

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

Although certain preferred examples and examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

Delivery systems as described herein may be used to position catheter tips and/or catheters to various areas of a human heart. For example, a catheter tip and/or catheter may be configured to pass from the right atrium into the coronary sinus. However, it will be understood that the description can refer or generally apply to positioning of catheter tips and/or catheters from a first body chamber or lumen into a second body chamber or lumen, where the catheter tips and/or catheters may be bent when positioned from the first body chamber or lumen into the second body chamber or lumen. A body chamber or lumen can refer to any one of a number of fluid channels, blood vessels, and/or organ chambers (e.g., heart chambers). Additionally, reference herein to “catheters,” “tubes,” “sheaths,” “steerable sheaths,” and/or “steerable catheters” can refer or apply generally to any type of elongate tubular delivery device comprising an inner lumen configured to slidably receive instrumentation, such as for positioning within an atrium or coronary sinus, including for example delivery catheters and/or cannulas. It will be understood that other types of medical implant devices and/or procedures can be delivered to the coronary sinus using a delivery system as described herein, including for example ablation procedures, drug delivery and/or placement of coronary sinus leads.

What is claimed is:

1. A medical implant for managing blood flow through a blood vessel, the medical implant comprising:
a stent body forming a proximal portion and a tapered distal end, the tapered distal end being angled relative to the proximal portion to face a direction of blood flow and limit blood flow into an inner lumen of the stent body, and the tapered distal end having a flexible structure to allow the tapered distal end to bend and straighten in response to blood flow; and
a covering extending at least partially along the stent body.

2. The medical implant of claim 1, wherein the stent body is sized for placement within an inferior vena cava.

3. The medical implant of claim 1, wherein the tapered distal end forms an orifice into the inner lumen of the stent body.

4. The medical implant of claim 3, wherein the orifice is at a central position of the tapered distal end.

5. The medical implant of claim 3, wherein bending of the tapered distal end reduces a size of the orifice in response to increased blood pressure through the blood vessel.

6. The medical implant of claim 3, wherein the tapered distal end is flexible to allow the tapered distal end to fully close the orifice in response to increased blood pressure through the blood vessel.

7. The medical implant of claim 1, wherein the covering comprises one or more bypass apertures to allow blood flow through the covering.

8. The medical implant of claim 1, wherein the tapered distal end comprises two or more overlapping leaflets.

9. The medical implant of claim 1, further comprising a plug coupled to the stent body via a tether, the plug being sized to fit into and cover an orifice formed by the tapered distal end.

10. The medical implant of claim 9, wherein the tether is shape-set to hold the plug distally from the stent body and outside the inner lumen of the stent body.

11. The medical implant of claim 9, wherein the tether is flexible to allow the plug to move towards the stent body in response to increase blood pressure through the blood vessel.

12. A medical implant for managing blood flow through a blood vessel, the medical implant comprising:
a stent body forming a proximal portion and a distal end enclosing an inner lumen of the stent body; and
a plug coupled to the stent body via a tether, the plug being sized to fit into and cover an orifice formed by the distal end of the stent body.

13. The medical implant of claim 12, wherein the tether is shape-set to hold the plug distally from the stent body and outside the inner lumen of the stent body.

14. The medical implant of claim 12, wherein the plug comprises a conical proximal end.

15. The medical implant of claim 12, wherein the plug comprises a bowl forming a spherical cap extending towards the inner lumen of the stent body and a concave interior facing a direction of blood flow through the blood vessel.

16. The medical implant of claim 15, wherein the plug comprises crossing arms supporting the bowl.

17. The medical implant of claim 16, wherein the stent body comprises a stopper positioned to prevent at least a portion of the crossing arms from entering the inner lumen of the stent body.

18. A medical implant for managing blood flow through a blood vessel, the medical implant comprising:
a stent body forming a proximal portion and a distal end enclosing an inner lumen of the stent body;
a restrictor valve coupled to the stent body and disposed within the inner lumen of the stent body; and
a plug coupled to the restrictor valve via a tether, the tether being shape-set to hold the plug outside the inner lumen of the stent body.

19. The medical implant of claim 18, wherein the tether is flexible to allow the plug to move into the inner lumen of the stent body in response to blood flow against the plug.

20. The medical implant of claim 18, wherein the tether comprises a coiled wire.
